SEQUENCE LISTING (22 sequences)

SEQ ID NO: 1 - protein, 652 aa, synthetic construct
  source 1..652: note = SR1
METDTLLLWVLLLWVPGSTGDQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSHHHHHH

SEQ ID NO: 2 - protein, 652 aa, synthetic construct
  source 1..652: note = SR2
METDTLLLWVLLLWVPGSTGDELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 3 - protein, 657 aa, synthetic construct
  source 1..657: note = SR3
METDTLLLWVLLLWVPGSTGDEVQLVESGGGLVQAGGSLRLSCAASGRTFSSYVMGWFRQATGKEREFVATIAWDSGSTYYADSVKGRFTISRDNAKNTVHLQMNSLKPEDTAVYYCAASYNVYYNNYYYPISRDEYDYWGQGTQVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSHHHHHH

SEQ ID NO: 4 - protein, 657 aa, synthetic construct
  source 1..657: note = SR4
METDTLLLWVLLLWVPGSTGDELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSEVQLVESGGGLVQAGGSLRLSCAASGRTFSSYVMGWFRQATGKEREFVATIAWDSGSTYYADSVKGRFTISRDNAKNTVHLQMNSLKPEDTAVYYCAASYNVYYNNYYYPISRDEYDYWGQGTQVTVSSHHHHHH

SEQ ID NO: 5 - protein, 523 aa, synthetic construct
  source 1..523: note = SR5
METDTLLLWVLLLWVPGSTGDELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 6 - protein, 447 aa, synthetic construct
  source 1..447: note = SR6
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 489 aa, synthetic construct
  source 1..489: note = SR7
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 591 aa, synthetic construct
  source 1..591: note = SR8
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 9 - protein, 633 aa, synthetic construct
  source 1..633: note = SR9
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 10 - protein, 633 aa, synthetic construct
  source 1..633: note = SR21
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 11 - protein, 518 aa, synthetic construct
  source 1..518: note = SR22
MALPVTALLLPLALLLHAARPEVQVVESGGGTVQAGGSLRLSCAASARTFANAHMAWFRQAPGKEREFLAAITWSGGITDYANSVKGRFTISRDNAENAMYLQMNSLKPEDTAIYICAVDTTSAYDQLWSRQSEYEYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 12 - protein, 518 aa, synthetic construct
  source 1..518: note = SR23
MALPVTALLLPLALLLHAARPEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 13 - protein, 775 aa, synthetic construct
  source 1..775: note = SR24
MALPVTALLLPLALLLHAARPEVQVVESGGGTVQAGGSLRLSCAASARTFANAHMAWFRQAPGKEREFLAAITWSGGITDYANSVKGRFTISRDNAENAMYLQMNSLKPEDTAIYICAVDTTSAYDQLWSRQSEYEYWGQGTQVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 14 - protein, 775 aa, synthetic construct
  source 1..775: note = SR25
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSEVQVVESGGGTVQAGGSLRLSCAASARTFANAHMAWFRQAPGKEREFLAAITWSGGITDYANSVKGRFTISRDNAENAMYLQMNSLKPEDTAIYICAVDTTSAYDQLWSRQSEYEYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 15 - protein, 775 aa, synthetic construct
  source 1..775: note = SR26
MALPVTALLLPLALLLHAARPEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 16 - protein, 775 aa, synthetic construct
  source 1..775: note = SR27
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 17 - protein, 389 aa, synthetic construct
  source 1..389: note = SR28
METDTLLLWVLLLWVPGSTGDSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 18 - protein, 518 aa, synthetic construct
  source 1..518: note = SR29
METDTLLLWVLLLWVPGSTGDSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 19 - protein, 518 aa, synthetic construct
  source 1..518: note = SR210
METDTLLLWVLLLWVPGSTGDQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNHHHHHH

SEQ ID NO: 20 - protein, 523 aa, synthetic construct
  source 1..523: note = SR211
METDTLLLWVLLLWVPGSTGDSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSEVQLVESGGGLVQAGGSLRLSCAASGRTFSSYVMGWFRQATGKEREFVATIAWDSGSTYYADSVKGRFTISRDNAKNTVHLQMNSLKPEDTAVYYCAASYNVYYNNYYYPISRDEYDYWGQGTQVTVSSHHHHHH

SEQ ID NO: 21 - protein, 523 aa, synthetic construct
  source 1..523: note = SR212
METDTLLLWVLLLWVPGSTGDEVQLVESGGGLVQAGGSLRLSCAASGRTFSSYVMGWFRQATGKEREFVATIAWDSGSTYYADSVKGRFTISRDNAKNTVHLQMNSLKPEDTAVYYCAASYNVYYNNYYYPISRDEYDYWGQGTQVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNHHHHHH

SEQ ID NO: 22 - DNA, 2519 bp, synthetic construct
  source 1..2519: mol_type = other DNA; note = mRNA IVT plasmid backbone
ggtttagtgaaccgtcagatccgctagagatccgcggccgctaatacgactcactataaggacatttgcttctgacacaactgtgttcactagcaacctcaaacagacaccgctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaccggtctagcataaccccttggggcctctaaacgggtcttgaggggttttttgcccgggggatcccctagggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttaaacggaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaaatgcggcctcgagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt

(19) United States
(12) Patent Application Publication
(10) Pub. No.: US 2026/0201419 A1
(43) Pub. Date: Jul. 16, 2026

(54) ARMED CAR-MACROPHAGE COMPOSITIONS AND METHODS OF USE THEREOF

(71) Applicant: RenaBio Therapeutics, Inc., Natick, MA (US)

(72) Inventors: Shengjun Ren, Newton, MA (US); Yu Zhang, Newton, MA (US)

(21) Appl. No.: 19/448,634

(22) Filed: Jan. 14, 2026

Related U.S. Application Data
(60) Provisional application No. 63/745,171, filed on Jan. 14, 2025.

(51) Int. Cl. C12N 15/88; A61K 9/51; A61K 31/7105; A61K 38/17; A61K 48/00; A61P 35/00; C07K 14/705; C12N 5/0786; C12N 5/10; C12N 15/11

(52) U.S. Cl. CPC C12N 15/88; A61K 9/5123; A61K 31/7105; A61K 38/1774; A61K 48/0033; A61K 48/005; A61P 35/00; C07K 14/70503; C12N 5/0645; C12N 5/10; C12N 15/11; C12N 2510/00

(57) ABSTRACT

A nanoparticle for selective transfection of M2 macrophages, the nanoparticle including a lipid phase and a core that contains one or more nucleic acids. The nanoparticle selectively delivers the one or more nucleic acids to M2 macrophages in vitro and in vivo. Also provided is a therapeutic composition that contains a plurality of the nanoparticle described above and a pharmaceutically acceptable excipient. Further provided are methods for selective transfection of M2 macrophages, for converting M2 macrophages into M1 macrophages, and for treating a cancerous tumor. The methods are carried out by contacting M2 macrophages with a composition that includes a plurality of the nanoparticle mentioned above.

### CROSS REFERENCE TO RELATED APPLICATIONS

This application claims the priority to and benefit of U.S. Provisional Application Ser. No. 63/745,171, filed Jan. 14, 2025, the content of which is hereby incorporated by reference in its entirety.

### REFERENCE TO AN ELECTRONIC SEQUENCE LISTING

A computer readable file containing a sequence listing is being electronically co-filed herewith via Patent Center. The computer readable file, submitted under 37 CFR § 1.831 (e), will also serve as the copy required by 37 § CFR 1.831 (c). The file (filename “RENA-001-101X.XML”) was created on Jan. 3, 2026, and has a size of 38,698 bytes. The content of the computer readable file is hereby incorporated by reference in its entirety.

### BACKGROUND

Adoptive cell therapy, such as chimeric antigen receptor (CAR) T cell therapy, has demonstrated great therapeutic efficacy in hematological malignancies. However, it has major drawbacks in treating solid tumors, in part, due to tumor microenvironment (TME) immunosuppression and antigen escape.

CAR T cell therapy requires a complicated and lengthy ex vivo manufacturing process. The extremely high cost of the therapy limits its accessibility to patients.

There is a critical need to develop adoptive cell therapy alternatives to traditional CAR T cell therapy, particularly for treating solid tumors, in the form of an off-the-shelf therapeutic product that has a lower cost and increased functionality that overcomes the drawbacks mentioned above.

### SUMMARY

To meet the above challenges, armed CAR-macrophage cell therapy is disclosed whose CAR gene, together with armed therapeutic modality genes are transferred into macrophage using an in vitro or in vivo (in situ) targetable nanoparticle to treat solid tumors.

To accomplish this therapy, a nanoparticle for selective transfection of M2 macrophages is disclosed. The nanoparticle includes a lipid phase and a core that contains one or more nucleic acids. The nanoparticle selectively delivers the one or more nucleic acids to M2 macrophages in vitro and in vivo.

Also provided is a therapeutic composition that contains a plurality of the nanoparticle described above and a pharmaceutically acceptable excipient.

Further provided is a method for selective transfection of M2 macrophages that is carried out by contacting M2 macrophages with a composition that includes a plurality of the nanoparticle mentioned above.

Also within the scope of the invention is a method for converting M2 macrophages into M1 macrophages. The method includes the step of contacting the M2 macrophages with a plurality of the nanoparticle set forth, supra.

Moreover, disclosed is a method for treating a cancerous tumor in a subject. The method comprising administering to the subject a composition comprising the nanoparticle mentioned above in which the one or more nucleic acids encode (i) a chimeric antigen receptor (CAR) that specifically binds to a tumor associated antigen (TAA), (ii) a T-cell engager (TCE) specifically binds to another TAA and binds to a T cell, and (iii) a therapeutic gene that is effective against the cancerous tumor.

The details of one or more embodiments of the invention are set forth in the description and drawings below. Other features, objects, and advantages of the invention will be apparent from the description, the drawings, and the claims.

### BRIEF DESCRIPTION OF THE DRAWINGS

The foregoing and other objects, features and advantages will be apparent from the following description of particular embodiments of the disclosure, as illustrated in the accompanying drawings.

FIG. 1 shows phase-contrast microscopy images of human monocytes (top left) and M0 macrophages (bottom left) obtained from monocytes treated with PMA and flow cytometry of the M0 macrophages (right).

FIG. 2 shows phase-contrast microscopy image of human M0 macrophages (top left) and M1 macrophages (bottom left) obtained from M0 macrophages treated with INF-γ and LPS, and flow cytometry of the M1 macrophages (right).

FIG. 3 is a bar graph showing secretion of IL-1β, IL-6, TNFα, and CXCL-10 by M1 macrophages.

FIG. 4 shows phase-contrast microscopy images of human M0 macrophages (top left) and M2 macrophages (bottom left) obtained from M0 macrophages treated with IL-4 and IL-13 and flow cytometry of the M2 macrophages (right).

FIG. 5 is a bar graph showing secretion of CCL-22 by M2 macrophages.

FIG. 6 shows fluorescence microscopy images of M2 macrophages observed 1 day after transfection with several GFP mRNA-LNP (LNP_A-E) of the invention (upper panels) and corresponding phase contrast microscopy images of the same cells (lower panels).

FIG. 7 shows fluorescence microscopy images of monocytes, M1 macrophages, and M2 macrophages observed 1 day after transfection with a GFP mRNA-LNP (LNP_B) of the invention (upper panels) and corresponding phase contrast microscopy images of the same cells (lower panels).

FIG. 8A shows fluorescence microscopy images of M2 macrophages observed 1 to 4 days after transfection with a GFP mRNA-LNP of the invention (upper panels) and corresponding phase contrast microscopy images of the same cells (lower panels).

FIG. 8B shows fluorescence microscopy images of M2 macrophages observed 5 to 8 days after transfection with a GFP mRNA-LNP of the invention (upper panels) and corresponding phase contrast microscopy images of the same cells (lower panels).

FIG. 9 shows fluorescence microscopy image of M2 macrophages observed 12 days after transfection with a GFP mRNA-LNP of the invention (left panel) and the corresponding phase contrast microscopy image of the same cells (right panel).

FIG. 10 is a bar graph showing luciferase activity in M2 macrophages transfected with a nanoparticle of the invention that contained mRNA encoding firefly luciferase (F-Luci), mRNA encoding GFP, or both mRNAs relative to background of untransfected M2 macrophages. Luciferase activity was measured at day 1 post-transfection (D1) and at day 4 post-transfection (D4) (n=12).

FIG. 11 shows fluorescence microscopy images of the same M2 macrophages shown in FIG. 10 observed 1 day (D1; top upper panels) and 4 days (D4; top lower panels) after transfection with a GFP mRNA-LNP of the invention and/or an LNP of the invention carrying a firefly luciferase (F-Luci) and corresponding phase contrast microscopy images of the same cells (bottom upper and lower panels).

FIG. 12 is a plot of TNFα secretion (pg/ml) from untransfected M0, M1, and M2 macrophages (1_M0, 4_M1, 6_M2, respectively) or M2 macrophages transfected with an LNP of the invention (12_M2_B300).

FIG. 13 shows bar graphs of luciferase activity (expressed as tumor burden relative to untreated HCC/M2 macrophage coculture) of hepatocellular carcinoma (HCC) HepG2 cells stably expressing luciferase and green fluorescent protein (HepG2_GFP/Luci) co-cultured with M2 macrophages untreated (M2) or treated with 1 μg/ml of the indicated CAR mRNA LNP. The luciferase activities were measured 1, 2, or 3 days after initiation of treatment. E/T=effector (M2) to target (HCC) ratio.

FIG. 14 shows bar graphs of Tumor burden relative to HepG2 control of HepG2_GFP/Luci cells co-cultured with M2 macrophages untreated (M2) or treated for 1 day with the indicated concentration of CAR mRNA LNP constructs at an E/T of 1/8.

FIG. 15 shows fluorescence microscopy images (upper panels) and corresponding phase contrast images (lower panels) of HepG2_GFP/Luci cells co-cultured with M2 macrophages untreated (left panels) or treated for 1 day with 1.5 μg/ml CAR^{SR9} mRNA LNP (right panels) at an E/T of 1/8.

FIG. 16 shows fluorescence microscopy images (upper panels) and corresponding phase contrast images (lower panels) of HepG2_GFP/Luci cells co-cultured with M2 macrophages untreated (left panels) or treated for 1 day with 3.0 μg/ml CAR^{SR9} mRNA LNP (right panels) at an E/T of 1/8.

FIG. 17 shows fluorescence microscopy images (upper panels) and corresponding phase contrast images (lower panels) of HepG2_GFP/Luci cells co-cultured with M2 macrophages untreated (left panels) or treated for 1 day with 6.0 μg/ml CAR^{SR9} mRNA LNP (right panels) at an E/T of 1/8.

FIG. 18 shows bar graphs of relative light units (RLU) of NFAT-Jurkat T cells (NFAT; T cells expressing luciferase under the control of the NFAT promoter) co-cultured with HepG2 cells and treated with supernatant from M2 macrophages (M2 control) or supernatant from M2 macrophages transfected with the indicated T cell engager mRNA LNP construct (TCE). Supernatant from transfected M2 was collected 1, 2, 5, or 7 days after TCE mRNA LNP treatment. Luciferase activities were measured 12 h after treatment. Background luciferase activity was measured from cultures of NFAT, HepG2, co-cultured NFAT and HepG2 (NFAT+HepG2), and medium alone.

FIG. 19 shows bar graphs as described in the legend for FIG. 18 except that the luciferase activity of NFAT-Jurkat cells co-cultured with HepG2 cells was measured 24 h after treatment.

FIG. 20 shows bar graphs of killing percentage of HepG2_GFP/Luci cells cocultured with T cells and treated with supernatant collected from M2 macrophages 1, 2, 5, or 7 days after transfection with the indicated TCE mRNA LNP. Killing percentage was calculated from luciferase activities of the indicated cultures compared to luciferase activity of HepG2_GFP/Luci cells cocultured with T cells treated with supernatant from untransfected M2 macrophages (0% killing). Luciferase activities were measured 48 h after treatment. The E/T ratio of the effective primary T cells to the targeting HCC HepG2 cancer cells was 1/2.

FIG. 21 shows bar graphs as described in the legend for FIG. 20 except that the luciferase activities were measured 72 h after treatment.

FIG. 22 shows bar graphs of luciferase activity (expressed as tumor burden compared to HepG2_GFP/Luci plus M2) of HepG2_GFP/Luci cells co-cultured with M2 macrophages and treated for 48 h with T cells and 1.0 μg/ml CAR^{SR9} mRNA LNP (CAR LNP) or 0.5 μg/ml TCE^{SR4} and CAR^{SR9} mRNA LNP (TCE_CAR LNP). The E/T for M2 was 1/8 and for T cells 1/4.

FIG. 23 shows bar graphs of luciferase activity (expressed as tumor burden relative to HepG2_GFP/Luci) of HepG2_GFP/Luci cells alone (HepG2) or HepG2_GFP/Luci cells co-cultured with M2 and treated for 24 h with T cells (Pan-T) alone (control) or T cells with the indicated amounts of TCE^{SR4} mRNA LNP (TCE_SR4) or TCE^{SR4}-CAR^{SR9} mRNA LNP (TCE_SR4-CAR_SR9). The E/T for M2 was 1/8 and for T cells 1/4.

FIG. 24 shows bar graphs of luciferase activity (expressed as tumor burden relative to HepG2_GFP/Luci+M2+T) of HepG2_GFP/Luci cells co-cultured with M2 and treated with T cells alone (control) or T cells with 2 μg/ml TCE^{SR4} (TCE LNP) or 2 g/ml TCE^{SR4}-CAR^{SR9} mRNA LNP (TCE-CAR LNP). Luciferase activity was measured 7 days after treatment. The E/T for M2 was 1/64 and for T cells 1/32.

FIG. 25 shows fluorescence microscopy images (upper panels) and corresponding phase contrast images (lower panels) of HepG2_GFP/Luci cells co-cultured with M2 macrophages and treated with T cells (left panels) or treated with T cells and 1.0 μg/ml TCE^{SR4}-CAR^{SR9} mRNA LNP (right panels). Images were obtained 3 days after treatment. The E/T for M2 was 1/8 and for T cells 1/2.

FIG. 26 shows fluorescence microscopy images (upper panels) and corresponding phase-contrast images (lower panels) obtained before and 6, 11, and 18 days after treatment of HepG2_GFP/Luci cells co-cultured with M2 macrophages and T cells with 0.8 μg/mL TCE^{SR4}_CAR^{SR9} mRNA LNP: The indicated cells (center lower panels) received a second dose of TCE^{SR4}_CAR^{SR9} mRNA LNP at day 11 and were imaged 7 days later. The right panel shows luciferase activity (expressed as tumor burden relative to that of 18 days post-treatment) for the indicated treatments. The E/T for M2 was 1/64 and the E/T for T cells was 1/32.

FIG. 27A shows flow cytometry plots of Pan T cells used in the studies described in FIGS. 25 and 26 above unstained (negative control) or stained with antibodies against CD45RO and CD3 (top right plot). CD45RO/CD3-positive cells were examined for CCR7 expression (bottom right plot). The CD45RO/CD3 positive cells are memory T cells (M.T.) and those cells included both central memory T cells (TCM) and effector memory T cells (TEM).

FIG. 27B shows flow cytometry plots of T cells recovered from the cultures of HepG2-GFP/Luci, M2, and Pan-T treated with TCE^{SR4}-CAR^{SR9} mRNA LNP described in FIG. 25. Abbreviations are shown in the legend to FIG. 27A.

FIG. 28 shows bar graphs summarizing the data from FIGS. 27A and 27B. Abbreviations are shown in the legends to FIGS. 25 and 26.

FIG. 29 shows bar graphs of luciferase activity (expressed as tumor burden relative to HepG2_GFP/Luci) of HepG2_GFP/Luci cells alone (HepG2), HepG2_GFP/Luci cells treated with T cells (Pan-T), and HepG2_GFP/Luci cells treated with MT cells generated in the TCE^{SR4}-CAR^{SR9} mRNA LNP treated co-culture study of HepG2_GFP/Luci cells+M2+T shown in FIGS. 24 and 25. SK_R1, SK_R2, and SK_R3 refer to rounds of treatment with MT serially collected from treated HepG2_GFP/Luci cells (R2 was treated with MT recovered from R1 and R3 was treated with MT recovered from R2).

FIG. 30 shows bar graphs of luciferase activity (expressed as tumor burden relative to HepG2_GFP/Luci) of HepG2_GFP/Luci (HepG2) and HepG2_GFP/Luci treated with T cells expanded from the corresponding co-cultures of HepG2_GFP/Luci/M2/TCE_SR4 and HepG2_GFP/Luci/M2/TCE_SR4-CAR_SR9 described in FIG. 23.

FIG. 31 is a diagram of the study design to examine in vivo pharmacological efficacy. NSG is NOD scid gamma mouse that is deficient in both B and T cells.

FIG. 32 is a plot of average tumor size versus days prior to or after the first of three injections close to or into the tumor of vehicle (control) or TCE^{SR4}_CAR^{SR9} mRNA LNP (TCE_CAR mRNA LNP) containing 4.5 μg each of TCE^{SR4} mRNA and CAR^{SR9} mRNA.

FIG. 33 is a plot of average body weight versus days following injection of TCE^{SR4}_CAR^{SR9} mRNA LNP or vehicle (control) as described in the legend to FIG. 32.

FIG. 34 is a plot of individual tumor size versus days prior to or after the first of three injections close to or into the tumor of vehicle (control) or TCE^{SR4}_CAR^{SR9} mRNA LNP as described in the legend to FIG. 32.

FIG. 35 shows flow cytometry analysis for the indicated markers of spleen cells collected from the cured cancer-free mice (5/7 mice showing no tumor growth in FIG. 34) in the left and center panels. The right panel is a bar graph showing average percentage of T cells expressing the indicated markers.

FIG. 36 is a plot of percent survival versus days post-injection of the mice described in the legend to FIG. 32.

FIG. 37 is a diagram of the study design to examine in vivo toxicology of the cancer treatment of the invention.

FIG. 38 is a plot of average tumor size versus days prior to or after the first of three injections near the tumor of vehicle (control) or TCE^{SR4}_CAR^{SR9} mRNA LNP (TCE_CAR mRNA LNP) containing 4.5 μg each of TCE^{SR4} mRNA and CAR^{SR9} mRNA.

FIG. 39 is a plot of average body weight versus days following injection of TCE^{SR4}_CAR^{SR9} mRNA LNP or vehicle (control) as described in the legend to FIG. 38.

FIG. 40A shows a bar graph of serum levels (units/L) of liver biomarker alanine aminotransferase (ALT) measured from mice with the indicated treatment. The horizontal dashed lines represent the normal range (NR) for the marker.

FIG. 40B shows a bar graph of serum levels (units/L) of liver biomarker aspartate aminotransferase (AST) measured from mice with the indicated treatment. The horizontal dashed lines are described in the legend to FIG. 40A.

FIG. 40C shows a bar graph of serum levels (mg/dL) of liver biomarker gamma-glutamyl transferase (GGT) measured from mice with the indicated treatment. The GGT levels were below the detection level of the assay (<7 mg/dL). The horizontal dashed lines are described in the legend to FIG. 40A.

FIG. 40D shows a bar graph of serum levels (mg/dL) of liver biomarker bilirubin measured from mice with the indicated treatment. The horizontal dashed lines are described in the legend to FIG. 40A.

FIG. 40E shows a bar graph of serum levels (units/L) of liver and kidney biomarker alkaline phosphatase (ALP) measured from mice with the indicated treatment. The horizontal dashed lines are described in the legend to FIG. 40A.

FIG. 40F shows a bar graph of serum levels (g/dL) of liver and kidney biomarker albumin measured from mice with the indicated treatment. The horizontal dashed lines are described in the legend to FIG. 40A.

FIG. 40G shows a bar graph of serum levels (mg/dL) of kidney biomarker blood urea nitrogen (BUN) measured from mice with the indicated treatment. The horizontal dashed lines are described in the legend to FIG. 40A.

FIG. 41A shows a bar graph of serum levels (units/L) of liver biomarker alanine aminotransferase (ALT) measured from the cured cancer-free mice (the 5/7 mice showing no tumor growth in FIG. 21 (TCE-CAR-LNP in situ)) and from NSG mice. The horizontal dashed lines are described in the legend to FIG. 40A.

FIG. 41B shows a bar graph of serum levels (units/L) of liver biomarker aspartate aminotransferase (AST) measured from the cancer-free mice and from NSG mice. The horizontal dashed lines are described in the legend to FIG. 40A.

FIG. 41C shows a bar graph of serum levels (mg/dL) of liver biomarker gamma-glutamyl transferase (GGT) measured from the cancer-free mice and from NSG mice. The GGT levels were below the detection level of the assay (<7 mg/dL). The horizontal dashed lines are described in the legend to FIG. 40A.

FIG. 41D shows a bar graph of serum levels (mg/dL) of liver biomarker bilirubin measured from the cancer-free mice and from NSG mice. The horizontal dashed lines are described in the legend to FIG. 40A.

FIG. 41E shows a bar graph of serum levels (units/L) of liver and kidney biomarker alkaline phosphatase (ALP) measured from the cancer-free mice and from NSG mice. The horizontal dashed lines are described in the legend to FIG. 40A.

FIG. 41F shows a bar graph of serum levels (g/dL) of liver and kidney biomarker albumin measured from the cancer-free mice and from NSG mice. The horizontal dashed lines are described in the legend to FIG. 40A.

FIG. 41G shows a bar graph of serum levels (mg/dL) of kidney biomarker blood urea nitrogen (BUN) measured from mice with the indicated treatment. The horizontal dashed lines are described in the legend to FIG. 40A.

FIG. 42 shows bar graphs of relative light units (RLU) of NFAT-Jurkat T cells (NFAT; T cells expressing luciferase under the control of the NFAT promoter) co-cultured with U87 glioblastoma cells (U87) and assayed 10 h after treatment with supernatant from M2 macrophages (negative control; NC) or supernatant from M2 macrophages transfected with the indicated T cell engager (TCE) mRNA lipid nanoparticle (LNP) construct. Supernatant from transfected M2 was collected 1, 2, 4, 7, or 10 days after TCE mRNA LNP transfection. Background luciferase activity was measured from cultures of U87 cells and co-cultures of NFAT cells and U87 cells.

FIG. 43 shows bar graphs of RLU of NFAT cells co-cultured with U251 glioblastoma cells (U251) and assayed 10 h after treatment with supernatant from M2 macrophages (negative control; NC) or supernatant from M2 macrophages transfected with the indicated TCE mRNA LNP construct. Supernatant from transfected M2 was collected 1, 2, 4, 7, or 10 days after TCE mRNA LNP treatment. Background luciferase activity was measured from cultures of U251 cells and co-cultures of NFAT cells and U251 cells.

FIG. 44 shows bar graphs of luciferase activity (expressed as tumor burden relative to untreated cells) of U138 glioblastoma cells stably expressing GFP and luciferase (U138_GFP/Luci) co-cultured with T cells assayed 3 days after treatment with supernatant from M2 macrophages transfected with the indicated TCE mRNA LNP construct (TCE). Supernatant from transfected M2 was collected 1, 2, 4, 7, or 10 days after TCE mRNA LNP treatment. The effector (T cell) to target (U138_GFP/Luci) ratio was 1/2.

FIG. 45 shows bar graphs of luciferase activity from a study similar to that shown in FIG. 44 except that U251 glioblastoma cells stably expressing GFP and luciferase (U251_GFP/Luci) was tested instead of U138_GFP/Luci.

FIG. 46 shows bar graphs of luciferase activity (expressed as tumor burden relative to glioblastoma cells+M2 macrophage+T cells) of U251_GFP/Luci cells co-cultured with M2 macrophages and T cells, either untreated (U251+M2+T) or treated with the indicated TCE mRNA LNP with the indicated E/T ratios (T cells to U251_GFP/Luci cells). Luciferase activities were determined 6 days after treatment with the TCE mRNA LNP at 1 μg./ml. The M2/U251_GFP/Luci E/T ratio was 1/8.

FIG. 47 shows bar graphs of tumor burden (normalized to untreated glioblastoma cell/M2 macrophage coculture) of U87_GFP/Luci cocultured with M2 macrophages untreated (NC) and treated with 1 μg/ml of the indicated chimeric antigen receptor mRNA LNP (CAR mRNA LNP SR21-SR27). Luciferase activity was determined 4 days after treatment. The E/T (M2 to U87_GFP/Luci) was 1/8.

FIG. 48 shows fluorescence microscopy images (upper panels) and corresponding phase contrast images (lower panels) of U87_GFP/Luci cells cocultured with M2 macrophages treated with 1 μg/ml of CAR^{SR27} mRNA LNP (left panels) or untreated (right panels). The images (10× magnification) were obtained 4 days post-treatment. The E/T for M2 was 1/8.

FIG. 49 shows bar graphs of luciferase activity (expressed as tumor burden relative to U87_GFP/Luci+M2+/−T) of U87_GFP/Luci cells co-cultured with M2 with or without T cells untreated (NC), treated with 2 μg/ml CAR^{SR27} mRNA LNP (CAR_SR27), 2 μg/ml TCE^{SR212} mRNA LNP (TCE_SR212) or 2 μg/ml TCE^{SR212}-CAR^{SR27} mRNA LNP (TCE_SR212-CAR_SR27). Luciferase activity was measured 3 days after treatment. The E/T for M2 was 1/64 and for T cells was 1/4.

FIG. 50 shows fluorescence microscopy images (upper panels) and corresponding phase contrast images (lower panels) of U87_GFP/Luci cells cocultured with M2 macrophages and T cells untreated (NC; right panels), treated with 2 μg/ml TCE^{SR212}-CAR^{SR27} mRNA LNP (left panels), and treated with 2 μg/ml TCE^{SR212} mRNA LNP (center panels). The images (10× magnification) were obtained 3 days post-treatment. The E/T for M2 was 1/64 and for T cells 1/4.

FIG. 51 shows bar graphs from a study similar to that shown in FIG. 49 except that U251 GFP/Luci was tested instead of U87_GFP/Luci. Details are found in the legend to that figure.

FIG. 52 shows fluorescence microscopy images and corresponding phase contrast images from a study similar to that described in FIG. 50 except that U251_GFP/Luci was tested instead of U87 GFP/Luci.

### DETAILED DESCRIPTION

As mentioned in the SUMMARY section, a nanoparticle for selective transfection of M2 macrophages is disclosed. The nanoparticle includes a lipid phase and a core that contains one or more nucleic acids.

The lipid phase includes an ionizable lipid, cholesterol, a phospholipid and a polyethylene glycol-conjugated lipid (pegylated lipid).

Ionizable lipids refer to a lipid or lipid-like material capable of being positively charged and able to electrostatically bind nucleic acids. As used herein, a “cationic lipid” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl, or more acyl chains, and the head group of the lipid typically carries the positive charge. Exemplary cationic lipids include one or more amine group(s) which bear the positive charge. Cationic lipids may encapsulate negatively charged RNA. In some aspects, cationic lipids are ionizable such that they may exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “ionizable” lipids or lipid-like materials are comprised by the term “cationic lipid” or “cationic lipid-like material” unless contradicted by the circumstances. Examples of cationic lipids include, but are not limited to: ((4-hydroxybutyl) azanediyl)bis(hexane-6,1-diyl)bis(2-hexyldecanoate), 1,2-dioleoyl-3-trimethylammonium propane (DOTAP), N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl) cholesterol (DC-Chol), dimethyldioctadecylammonium (DDAB), 1,2-dioleoyl-3-dimethylammonium-propane (DODAP), 1,2-diacyloxy-3-dimethylammonium propanes, 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy) propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleoyloxypropyl-3-dimethyl-hydroxyethyl ammonium bromide (DORIE), 2,3-dioleoyloxy-N-[2 (spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy) propane (CLinDMA), 2-[5′-(cholest-5-en-3-beta-oxy)-3′-oxapentoxy)-3-dimethyl-1-(cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (+)-N-(3-aminopropyl)-N,N-dimethyl-2,3-bis(cis-9-tetradecenyloxy)-1-propanaminium bromide (GAP-DMORIE), (+)-N-(3-aminopropyl)-N,N-dimethyl-2,3-bis(dodecyloxy)-1-propanaminium bromide (GAP-DLRIE), (+)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy) propan-1-aminium (DOBAQ), 2-({8-[(3b)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl)thio)-carbonyl) azanediyl)dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), Di((Z)-non-2-en-1-yl)-9-((4-(dimethylaminobutanoyl)oxy) heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino]-ethylamino) propionamide (lipidoid 98N12-5), 1-[2-[bis(2-hydroxydodecyl)-amino]ethyl-[2-[4-[2-[bis(2 hydroxydodecyl)amino]ethyl]piperazin-1-yl]ethyl]-amino]dodecan-2-ol (lipidoid 02-200); C12-200; or heptadecan-9-yl 8-((2-hydroxyethyl) (6-oxo-6-(undecyloxy) hexyl)amino) octanoate (SM-102). In some aspects, 1, 2, 3, 4, 5, or more of the foregoing cationic lipids may be excluded from the LNPs of the present disclosure.

Preferably, the ionizable lipid is (6Z,9Z,28Z,31Z)-heptatriaconta-6,9,28,31-tetraen-19-yl 4-(dimethylamino)butanoate (D-Lin-MC3-DMA), heptadecan-9-yl 8-((2-hydroxyethyl) (6-oxo-6-(undecyloxy) hexyl)amino) octanoate (SM102), 9Z,12Z-octadecadienoic acid, 3-[4,4-bis(octyloxy)-1-oxobutoxy]-2-[[[[3-(diethylamino) propoxy]carbonyl]oxy]methyl]propyl ester (LP01), 6-((2-hexyldecanoyl)oxy)-N-(6-((2-hexyldecanoyl)oxy) hexyl)-N-(4-hydroxybutyl) hexan-1-aminium (ALC-0315), and analogs thereof. Analogs of the ionizable lipids include modifications of one or more of the head, chemical linkage, bridge chain, and tail portion.

The chemical nature changes of the ionizable lipid's head amine group, e.g., substituting different amine groups such as tertiary amines, secondary amines, or cyclic amines, can affect its pKa value, which impacts how well the head interacts with nucleic acids inside an acidic endosome.

The ionizable lipid's chemical linkage (bridge chain) connects the head group to the tail. Its alteration, e.g., introducing ester, amide, ether, or disulfide linkages, can influence the degradation rate and stability of the ionizable lipid. This alteration can also affect endosomal escape.

The modification of the ionizable lipid tail includes using different fatty acid chains with variations of chain lengths, unsaturation degree, and/or branching patterns. It can impact the ionizable lipid overall hydrophobicity and membrane-disrupting capabilities.

The ionizable lipid typically constitutes by mole percentage 5-95% (e.g., 37% to 47%, 10-90%, 15-85%, 20-80%, 25-70%, 30-60%, 35-50%, 38%, 39%, 40%, 41%, 42%, 43%, 44%, 45%, and 46%). Further a molar ratio of the ionizable lipid to the one or more nucleic acids is preferably in the range of 2:1 to 6:1 (e.g., 2.5:1 to 5.9:1, 3:1 to 5.8:1, and 3.3:1 to 5.6:1).

The nanoparticle further contains cholesterol or cholesterol derivative. Examples of cholesterol derivatives include, but are not limited to, cholestanol, 5,6-epoxy cholestanol, cholestanone, cholestenone, coprostanol, cholesteryl-2′-hydroxyethyl ether, cholesteryl-4′-hydroxybutyl ether, 24-ethyl cholesterol, 24-methyl cholesterol, cholenic Acid, 3-hydroxy-5-cholestenoic Acid, cholesteryl palmitate, cholesteryl arachidonate, cholesteryl arachidate, cholesteryl myristate, cholesteryl palmitoleate, cholesteryl lignocerate, cholesteryl oleate, cholesteryl stearate, cholesteryl erucate, cholesterol α-linolenate, cholesteryl linoleate, cholesteryl homo-γ-linolenate, 4-hydroxy cholesterol, 6-hydroxy cholesterol, 7-hydroxy cholesterol, 19-hydroxy cholesterol, 20-hydroxy cholesterol, 22-hydroxy cholesterol, 24-hydroxy cholesterol, 25-hydroxy cholesterol, 27-hydroxy cholesterol, 27-alkyne cholesterol, 7-keto cholesterol, 7-dehydro cholesterol, 8-dehydro cholesterol, 24-dehydro cholesterol, 5α-hydroxy-6-keto cholesterol, 20,22-dihydroxy cholesterol, 7,25-dihydroxy cholesterol, 7,27-dihydroxy cholesterol, 7-keto-25-hydroxy cholesterol, fucosterol, phytosterol, cholesteryl 11,14-eicosadienoate, dimethyl hydroxyethyl aminopropane carbamoyl cholesterol iodide and mixtures thereof. The cholesterol derivative may comprise a sugar moiety and/or amino acids. In an embodiment the amino acids are selected from serine, threonine, lysine, histidine, arginine or their derivatives.

The nanoparticle includes the cholesterol or cholesterol derivative in an amount typically ranging by mole percentage 1% to 90% (e.g., 42% to 50%, 5% to 85%, 10% to 80%, 15% to 75%, 20% to 70%, 25% to 65%, 30% to 60%, 35% to 55%, 43%, 44%, 45%, 46%, 47%, 48%, and 49%).

Exemplary phospholipids include phosphatidylcholines, e.g., diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), dioleoylphosphatidylglycerol (DOPG), dipalmitoylphosphatidylglycerol (DPPG), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), and 1-hexadecyl-sn-glycero-3-phosphocholine (C16 Lyso PC); and phosphatidylethanolamines, e.g., diacylphosphatidylethanolamines, such as dioleoyl-phosphatidylethanolamine (DOPE), 1,2-diundecanoyl-sn-glycero-phosphocholine (DUPC), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dilauroyl-phosphatidylethanolamine (DLPE), distearoyl-phosphatidylethanolamine (DSPE), 1-phytanoyl-phosphatidylethanolamine (DpyPE), 16-O-monomethyl PE, 16-O-dimethyl PE, 18-1-trans PE, 1-stearoyl-2-oleoylphosphatidyethanolamine (SOPE), 1,2-dielaidoyl-sn-glycero-3-phosphoethanolamine (transDOPE), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some aspects, 1, 2, 3, 4, 5, or more of the foregoing neutral lipids may be excluded from the LNPs of the present disclosure. Without being bound by any theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some aspects, the molar ratio of the cationic lipid to the neutral lipid ranges from or from about 2:1 to about 8:1, or from or from about 10:0 to about 1:9, about 4:1 to about 1:2, or about 3:1 to about 1:1. In another aspect, the molar ratio of the cationic lipid to cholesterol ranges from or from about 2:1 to 1:1. In a further aspect, the molar ratio of the cationic lipid to the pegylated lipid ranges from or from about 100:1 to about 10:1 or from about 100:1 to about 25.1. In some aspects, the non-cationic lipid, e.g., neutral lipid (e.g., one or more phospholipids and/or cholesterol), may comprise from or from about 0 mol % to about 90 mol %, from or from about 0 mol % to about 80 mol %, from or from about 0 mol % to about 70 mol %, from or from about 0 mol % to about 60 mol %, or from or from about 0 mol % to about 50 mol %, of the total lipid present in the particle. In some aspects, the non-cationic lipid, e.g., neutral lipid (e.g., one or more phospholipids and/or cholesterol), may or may not be at least, at most, exactly, or between (inclusive or exclusive) of 0 mol %, 10 mol %, 20 mol %, 30 mol %, 40 mol %, 50 mol %, 60 mol %, 70 mol %, 80 mol %, or 90 mol % of the total lipid present in the particle. In some aspects, the phospholipid is in the range, by molar percentage, of 1% to 30% (e.g., 7% to 12%, 2% to 25%, 3% to 20%, 4% to 18%, 5% to 15%, 8%, 9%, 10%, and 11%).

Preferred phospholipids include dilinoleoylphosphatidylcholine (DLPC), dipalmitoylphosphatidylcholine (DPPC), distearoylphosphatidic acid (DSPA), dipalmitoylphosphatidic acid (DPPA), distearoylphosphatidylcholine (DSPC), dimyristoylphosphatidylethanolamine (DMPE), diheptanoylphosphatidylcholine (DHPC), dimyristoylphosphatidylcholine (DMPC), stearoylpalmitoylphosphatidyl-choline (SPPC), and diarachidoylphosphatidylcholine (DAPC).

Further, the pegylated lipids include polyethylene glycol (PEG)-lipid conjugates, such as PEG coupled to lipids (for example, DMG-PEG 2000), PEG coupled to phospholipids (for example, phosphatidylethanolamine (PEG-PE)), PEG conjugated to cholesterol or a derivative thereof, and mixtures thereof. In certain instances, PEG may be optionally substituted by alkyl, alkoxy, acyl, or aryl group.

Useful PEGs in the PEG-lipid conjugates are classified by their molecular weights; for example, PEG 2000 has an average molecular weight of about 2,000 Daltons, and PEG 5000 has an average molecular weight of about 5,000 Daltons. PEGs are commercially available from suppliers such as Avanti Polar Lipids. The PEG moiety of the PEG-lipid conjugates described herein may comprise a number-average molecular weight ranging from 550 Daltons to 20,000 Daltons (e.g., 600 Daltons to 15,000 Daltons, 800 Daltons to 10,000 Daltons, 900 Daltons to 8,000 Daltons, 1,000 Daltons to 6,000 Daltons, 1,200 Daltons to 5,000 Daltons, 1,300 Daltons to 4,000 Daltons, 1,400 Daltons to 3,500 Daltons, 1,500 Daltons to 3,000 Daltons, 1,600 Daltons, 1,800 Daltons, 1,900 Daltons, 2,000 Daltons, 2,100 Daltons, 2,200 Daltons, and 2,500 Daltons).

Suitable lipids for conjugating with PEG include phosphatidylethanolamines, diacylglycerols, phosphatidic acids, ceramides, dialkylamines, dialkylglycerols, 1,2-diacyloxypropan-3-amines, and mixtures thereof. These lipids have a variety of acyl chain groups of varying chain lengths and degrees of saturation. They are commercially available or can be isolated or synthesized using conventional techniques. The lipids can comprise saturated or unsaturated fatty acids with carbon chain lengths in the range of C10 to C20. They can contain mono- or polyunsaturated fatty acids and mixtures of saturated and unsaturated fatty acids. The lipids contemplated include, but are not limited to, dimyristoylglycerol (DMG), dimyristoylphosphatidylethanolamine (DMPE), dipalmitoyl-phosphatidylethanol amine (DPPE), dioleoylphosphatidylethanolamine (DOPE), and distearoylphosphatidylethanolamine (DSPE).

Suitable pegylated lipids are known in the art, e.g., WO2025238563A1. Examples include distearoylphosphatidylethanolamine-polyethylene glycol 2000 (DSPE-PEG 2000), dimyristoyl-PEG 2000 (DMG-PEG 2000), DSPE-PEG 2000-Mannose, DMG-PEG 2000-Mannose, PEG-c-DOMG, PEG-DLPE, PEG-DMPE, PEG-DPPC, PEG-ceramide conjugates (e.g., PEG-CerCI4 or PEG-CerC20), and PEG-c-DOMG. The pegylated lipid constitutes by molar percentage from 0.1% to 30% (e.g., 1.5% to 5.5%, 0.2% to 25%, 0.3% to 20%, 0.4% to 15%, 0.5% to 10%, 0.8% to 8%, 1% to 7%, 2%, 2.5%, 3%, 3.5%, 4%, 4.5%, and 5%).

In certain embodiments, the lipid phase includes at least two pegylated lipids selected from DSPE-PEG 2000, DMG-PEG-2000, DSPE-PEG 2000-Mannose, and DMG-PEG-2000-Mannose.

The mole percentage of the ionizable lipid, the cholesterol, the phospholipid, and the pegylated lipid in the nanoparticle is 37% to 47%, 42% to 50%, 7% to 12%, and 1.5% to 5.5%, respectively.

As also mentioned above, the nanoparticle includes a core containing one or more nucleic acid. The core can include a buffer solution. The buffer can be at a pH of 4.5 to 6.5 at a concentration of 25 mM to 300 mM. Exemplary buffers include sodium citrate buffer and sodium acetate buffer.

The nanoparticle has a molar ratio of the ionizable lipid to the one or more nucleic acids ranging from 2:1 to 6:1, preferably 3:1 to 6:1, more preferably 3.3:1 to 5.6:1.

The one or more nucleic acid in the nanoparticle of the invention can encode a chimeric antigen receptor (CAR), a T-cell engager (TCE), and/or a therapeutic gene. In a particular embodiment, the nucleic acid is an mRNA molecule encoding the CAR TCE, or therapeutic gene. In a specific embodiment, the CAR, TCE, and therapeutic gene are encoded by separate mRNA molecules. Alternatively, a single mRNA molecule can encode the CAR, TCE, and therapeutic gene. In a preferred embodiment, the nanoparticle includes two individual mRNA molecules, one encoding the CAR and the other encoding the TCE.

Not to be bound by theory, the combination of the CAR and the TCE will expand the number of tumor-associated antigen (TAA) targets to overcome solid tumor associated genetic heterogeneity and therapy-induced targets loss. In addition, the activation and mobilization of the bystander T cells in the TME by the TCE will cross talk with macrophage to initiate neoantigen cancer vaccine in the treated solid tumor TME. It will alleviate TME immunosuppression and result in long term therapeutic efficacy.

The CAR includes a binding domain that specifically recognizes a TAA. The TAA can be, but is not limited to, colon cancer antigen 19.9, cancer antigen B1, B7 H3, beta-catenin, blood group ALeb/Ley, Burkitt's lymphoma antigen-38.13, colonic adenocarcinoma antigen C14, ovarian carcinoma antigen CA125, Carboxypeptidase M, CD5, CD19, CD20, CD22, CD23, CD25, CD27, CD30, CD33, CD36, CD45, CD46, CD52, CD79a/CD79b, CD103, CD317, CD133, a gastric cancer mucin, antigen 4.2, glycoprotein A33 (gpA33), ADAM-9, gastric cancer antigen AH6, Claudi 18.2, Claudi 6, ALCAM, malignant human lymphocyte antigen APO-1, carcinoembryonic antigen (CEA); CEACAM5, CEACAM6, C017-iA, CO-43 (blood group Leb), CO-514 (blood group Lea), CTA-1, CTLA4, Cytokeratin 8, antigen D1.1, antigen D 156-22, DR5, Ei series (blood group B), EGFR (Epidermal Growth Factor Receptor), Ephrin receptor A2 (EphA2), ErbB1, ErbB3, ErbB4, GAGE-1, GAGE-2, Interleukin-13 Receptor a2 (IL13Ra2), JAM-3, KID3, KID31, KS 1/4 pan carcinoma antigen, human lung carcinoma antigens L6 and L20, LEA, LUCA-2, antigen FC10.2, G49, ganglioside GD2, ganglioside GD3, ganglioside GM2, ganglioside GM3, GD2, GD3, GICA 19-9, GM2, gpOO, glypican-3 (GPC3), EpCAM, human leukemia T cell antigen Gp37, melanoma antigen gp75, gpA33, HER2 antigen, human milk fat globule antigen (HMFG), human papillomavirus E6/human papillomavirus-E7, high molecular weight melanoma antigen (HMW MAA), I antigen (differentiation antigen) I(Ma), Integrin Alpha-V-Beta-6 IntegrinP6 (ITGB6), Mi:22:25:8, M18, M39, lung adenocarcinoma antigen F3, DLL3, MAGE-1, MAGE-3, MART, MUC-1, MUM-1, Myl, N acetylglucosaminyltransferase, neoglycoprotein, NS-10, OFA-1, OFA-2, Oncostatin M p15, melanoma-associated antigen p97, polymorphic epithelial mucin (PEM), TNF-alpha receptor, TNF-beta receptor, TNF-gamma receptor, TRA-1-85 (blood group H), Transferrin Receptor, tumor-specific transplantation antigen (TSTA), oncofetal antigen-alpha-fetoprotein (AFP), VEGF, VEGFR, VEP8, VEP9, VIM-D5, and Y hapten Ley, polymorphic epithelial mucin antigen (PEMA), PIPA, prostate-specific antigen (PSA), prostate-specific membrane antigen (PSMA), prostatic acid phosphate R2 4, RORi, sphingolipids, SSEA-1, SSEA-3, SSEA-4, sTn, T cell receptor derived peptide vT 5A7, TAG-72, and TL5 (blood group A).

In some embodiments, the CAR also includes a CD8a signal peptide, a CD8a hinge, a CD28 transmembrane domain, a CD32 signaling domain, and/or a 4-1BB costimulatory domain, and/or a TRL4-TIR domain, or combinations of these domains.

The CAR can have (include or consist of) the amino acid sequence of any one of SEQ ID NOs 6 to 16 or amino acid sequences having 75% to 99% similarity (75%, 80%, 85%, 90%, 95%, 99%) to SEQ ID NOs 6 to 16. Nucleic acids encoding these CAR are also within the scope of the invention.

The TCE can include a binding domain that specifically binds to a TAA and a binding domain that specifically binds to T cells via CD3 or CD137 (4-1BB). In certain embodiments, the TCE includes two TAA binding domains and a T cell binding domain.

The TAA binding domain of the TCE can be, but is not limited to, CEA, GPC3, MUC-1, EpCAM, HER receptors, PEM, A33, G250, carbohydrate antigens Ley, Lex, Leb, CD24, CD44, E-cadherin, SPARC, ErbB2, ErbB3, WT1, MUC1, LMP2, HPV E6&E7, EGFR, EGFRVIII, HER-2/neu, MAGE A3, p53 nonmutant, p53 mutant, NY-ESO-1, GD2, PSMA, PCSA, Interleukin-13 Receptor a2 (IL13Ra2), MelanA/MART1, Ras mutant, proteinase3 (PR1), bcr-abl, tyrosinase, survivin, PSA, hTERT, TAG-72, STEAP1, CD133, and CD166.

The TCE can have (include or consist of) the amino acid sequence of any one of SEQ ID NOs 1 to 5 and 17-21 or amino acid sequences having 75% to 99% similarity (75%, 80%, 85%, 90%, 95%, 99%) to SEQ ID NOs 1 to 5 and 17 to 21. Nucleic acids encoding these TCE are also within the scope of the invention.

As mentioned above, the mRNA can also encode a therapeutic gene. The therapeutic gene provides additional anti-cancer efficacy to the therapy. The therapeutic gene can be, e.g., an anti-VEGF antibody, an anti-VEGFR antibody, IL-2, IL-2 muteins, TGFβ-trap, TGFβ receptor muteins, anti-PD-1 antibody, anti-PD-L1 antibody, and anti-CD47 antibody.

The following method can be used to prepare the nanoparticle of the invention. The lipid phase components are mixed together at room temperature in a solvent, e.g., absolute ethanol, in the molar percentages set forth, supra. Separately, the one or more nucleic acid is formulated, also at room temperature, in a solution containing a buffer, e.g., sodium acetate buffer, to stabilize the nucleic acid. The lipid phase and solution are then mixed using an automated mixing instrument in a 1:1 to 1:5 v/v ratio of lipid phase to solution with a flow rate of 2 to 100 mL per minute at room temperature.

A method for selective transfection of M2 macrophages is also provided. The method is carried out by contacting M2 macrophages with a composition that includes the nanoparticle set out above. The nanoparticle can enclose one or more nucleic acids that encode a CAR, a TCE, and/or a therapeutic protein. In an embodiment, the one or more nucleic acids are mRNA molecules. The contacting step above can be carried out in vitro or in vivo. If carried out in vivo, the composition is delivered into a tissue that contains M2 macrophages, e.g., a solid tumor. The tumor can be malignant.

Also within the scope of the invention is a therapeutic composition that contains a plurality of the nanoparticle described above, together with a pharmaceutically acceptable excipient, e.g., solvents, salts, and sugars/cryoprotectants.

Another method is provided for converting M2 macrophages into M1 macrophages by contacting the M2 macrophages with a plurality of the above-described nanoparticle. The contacting step, like that in the above method for selective M2 macrophage transfection, can be carried out in vitro or in vivo. Again, if carried out in vivo, the composition is delivered into a tissue that contains M2 macrophages.

As summarized above, also disclosed is a method for treating a cancerous tumor in a subject by administering a nanoparticle of the invention to a subject suffering from cancer. The nanoparticle includes one or more nucleic acids that encode a CAR, a TCE, and, optionally, a therapeutic gene. The CAR, TCE, and therapeutic gene are those described above.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications, including patent documents, cited herein are incorporated by reference in their entirety.

### EXAMPLES

### Materials and Methods

TABLE 1
Source of materials

| Catalog # | Source | Description |
|---|---|---|
| 200-02-1MG | Peprotech | Recombinant Human IL-2 |
| BE02-053Q | Lonza | X-VIVOTM 15 Serum-free |
| | | Hematopoietic Cell Medium |
| 7930 | StemCell | CryoStor ® CS10 |
| | Technologies | |
| EDK001 | Kerafast | U251 Cell Line human |
| 12648010 | ThermoFisher | Gibco Recovery ™ Cell Culture Freezing |
| | | Medium |
| 30-2020 | ATCC | Fetal Bovine Serum |
| 11132D | ThermoFisher | Dynabeads ™ Human T-Activator |
| | | CD3/CD28 for T Cell Expansion and |
| | | Activation |
| 31-985-062 | ThermoFisher | Gibco ™ Opti-MIEM ™ I Reduced Serum |
| | | Medium |
| 30-2005 | ATCC | IMDM |
| 324404 | Biolegend | FITC anti-human CD340 (erbB2/HER-2) |
| | | Antibody |
| 352904 | Biolegend | PE anti-human EGFR Antibody |
| 100393-R024-A | SinoBiological | Anti-human GPC-3 antibody, APC |
| | | labeled |
| 354406 | Biolegend | APC anti-human CD213a2 (IL13Rα2) |
| | | Antibody |
| 17951 | StemCell | EasySep ™ Human T Cell Isolation Kit |
| | Technologies | |
| 19669 | StemCell | EasySep ™ Direct Human Monocyte |
| | Technologies | Isolation Kit |
| PLV-10172-50 | Cellomics | Firefly luciferase-GFP lentivirus (CMV, |
| | Technology | Puro) (2 × 25 ul) |
| 10569044 | Thermofisher | Gibco DMEM, high glucose, |
| | | GlutaMAX ™ Supplement, pyruvate |
| 20144 | StemCell | EasySep ™ Buffer |
| | Technologies | |
| 60621 | BPS Bioscience | NFAT Reporter (Luc) - Jurkat Cell line |
| IL2-HF2H3- | Acrobiosystems | FITC-Labeled Human IL-13 R alpha 2 |
| 25 ug-290 | | Protein, His Tag |
| 100-1061 | StemCell | CryoStor ® CS10 |
| | Technologies | |
| 10971 | StemCell | ImmunoCult ™ Human CD3/CD28 |
| | Technologies | |
| 10981 | StemCell | ImmunoCult ™-XF T Cell Expansion |
| | Technologies | Medium |
| 78036.1 | StemCell | Human Recombinant IL-2 (CHO- |
| | Technologies | expressed) |
| Catalog | BPS Bioscience | ONE-Step ™ Luciferase Assay System |
| #60690-1 | | |
| HE2-HF224- | Acrobiosystems | FITC-Labeled Human Her2/ErbB2 |
| 25 ug | | Protein, His Tag |
| 352919 | BioLegend | BV711 anti-human EGFR |
| 354405 | BioLegend | APC anti-human CD213a2 (IL13Ra2) |
| 324420 | BioLegend | BV421 anti-human CD340 (erbB2/HER- |
| | | 2) |
| GP3-HF258 | Acro Biosystems | FITC-Labeled Human Glypican 3/ |
| | | GPC3 Protein, Fc Tag |
| A1049101 | ThermoFisher | RPMI-1640 Medium |
| 122799 | Revvity | XenoLight D-Luciferin - K+ Salt |
| | | Bioluminescent Substrate |
| 317344 | Biolegend | BV421 anti-human CD3 Antibody |
| 89081402-1VL | Sigma | U-87 Cell Line human |
| HB-8065 | ATCC | HepG2 HCC Cell line |
| L00436 | GenScript | His Tag ELISA Detection Kit |
| 60690-2 | BPS Bioscience | ONE-Step ™ Luciferase Assay System |
| LS-F55748-1 | LSBio | His Tag (Competitive EIA) ELISA Kit |
| HTB-16 | ATCC | U138 Cell Line Human |
| 60097FI | StemCell | Anti-Human CD45RO Antibody, Clone |
| | Technologies | UCHL1, FITC labeled |
| 100-0288 | StemCell | Anti-Human CD3 Antibody, Clone |
| | Technologies | OKT3, APC |
| 100-0316 | StemCell | Anti-Human CD45RA, Clone HI100 |
| | Technologies | APC labeled |
| 353214 | Biolegend | Anti-Human CD197 (CCR7) Antibody, |
| | | Clone G043H7, |
| | | APC labeled |

### Polarization of Human Macrophages

Human monocytes were induced to M0 first and then polarized to either M1 or M2 macrophages using related cytokine cocktails as shown in FIGS. 1, 2, and 4.

Production of mRNA LNP

CAR mRNA and TCE mRNA were synthesized by in vitro transcription (IVT) after inserting the corresponding cDNAs into the mRNA IVT construct backbone (SEQ ID NO: 22). The amino acid sequences encoded by these mRNAs are shown in the sequence listing as set out in Table 2 below.

TABLE 2
CAR and TCE specificities and corresponding SEQ ID NOs

| Construct | Specificities | SEQ ID NO |
|---|---|---|
| TCESR1 | anti-EGFR/EpCAM/CD3 | SEQ ID NO: 1 |
| TCESR2 | anti-EGFR/EpCAM/CD3 | SEQ ID NO: 2 |
| TCESR3 | anti-EGFR/EpCAM/CD3 | SEQ ID NO: 3 |
| TCESR4 | anti-EGFR/EpCAM/CD3 | SEQ ID NO: 4 |
| TCESR5 | anti-EpCAM/CD3 | SEQ ID NO: 5 |
| CARSR6 | GPC3 | SEQ ID NO: 6 |
| CARSR7 | GPC3 | SEQ ID NO: 7 |
| CARSR8 | GPC3 | SEQ ID NO: 8 |
| CARSR9 | GPC3 | SEQ ID NO: 9 |
| CARSR21 | anti-HER2 | SEQ ID NO: 10 |
| CARSR22 | anti-EGFR | SEQ ID NO: 11 |
| CARSR23 | anti-EGFR | SEQ ID NO: 12 |
| CARSR24 | anti-HER2/EGFR | SEQ ID NO: 13 |
| CARSR25 | anti-HER2/EGFR | SEQ ID NO: 14 |
| CARSR26 | anti-HER2/EGFR | SEQ ID NO: 15 |
| CARSR27 | anti-HER2/EGFR | SEQ ID NO: 16 |
| TCESR28 | anti-IL13Rα2/CD3 | SEQ ID NO: 17 |
| TCESR29 | anti-EGFR/IL13Rα2/CD3 | SEQ ID NO: 18 |
| TCESR210 | anti-EGFR/IL13Rα2/CD3 | SEQ ID NO: 19 |
| TCESR211 | anti-EGFR/IL13Rα2/CD3 | SEQ ID NO: 20 |
| TCESR212 | anti-EGFR/IL13Rα2/CD3 | SEQ ID NO: 21 |
| mRNA IVT Plasmid | N/A | SEQ ID NO: 22 |

IVT was performed with pseudo-uridine and a capping reagent. LNP lipid components were described above and in Table 3 below.

The mRNA LNPs were produced with the iNanno L as directed by the manufacturer (MicroNano) using its single molecule detector (SMD) card at room temperature. The mRNA LNPs were stored in 10% (w/v) sucrose at −80° C. The LNP particle size, surface charge and concentration were analyzed using Zeta Sizer Red.

### TCE Production by Human M2 Macrophages

Human M2 macrophages were treated with TCE mRNA LNP at a concentration of 1 μg/ml TCE mRNA. The supernatants were collected 1-, 2-, 4-, 7-, 10-days post of the treatment. The collected supernatants were used for the TCE activity assay (NFAT-Jurkat) and TCE-cytotoxicity assay described below at 50 μl of supernatant per well of a 96-well plate.

### In Vitro Cytotoxicity Assays

GFP and Luciferase-expressing HCC or GBM cancer cells at a concentration of 20,000, 40,000, 80,000, or 160,000 cells/well were plated in 96-well plates containing 2,500 or 5,000 induced human M2 macrophages. On the following day, mRNA LNPs at a concentration of mRNA at 0.5, 1.0, 2.0, or 2.5 g/ml and Pan-T cells at 1/2, 1/4, or 1/32 effector-to-target (E/T) ratios were added. Plates were incubated at 37° C. for 1, 2, 3, and 7 days and subsequently, D-firefly luciferin potassium salt was added to the wells, and luminescence was measured with a microplate reader. Target cells incubated without effector cells were used to measure spontaneous death and set the baseline measurement.

### T-Cell Activation by TCE

HCC or GBM cancer cells at a concentration of 20,000 cells/well were plated in 96-well plates, and on the next day, Jurkat (NFAT-Luciferase) reporter cells as well as TCEs collected from mRNA LNP treated M2 macrophage as supernatants (50 μl of supernatant per well) were added at an E/T ratio (Jurkat/HCC or GBM) of 1/2 for 10, 12, or 24 hrs. Luciferase activity was then assessed using ONE-Step™ Luciferase assay system and luminescence was measured in a microplate reader.

### Human Primary T Cell Isolation

Human Pan T-cells were isolated from frozen peripheral blood leukopaks obtained from consenting healthy blood donors (STEMCELL technologies) by negative selection using EasySep Human T cells isolation kit (STEMCELL technologies). Pan T cells were activated with anti-CD3/CD28 cocktail (STEMCELL technologies). The expanded T cells were collected and resuspended in cryopreservation medium (CryoStor® CS10, STEMCELL technologies), aliquoted and stored under liquid nitrogen.

### Flow Cytometry Analysis

For assessing target associated antigen (TAA) expression on HCC and GBM cell lines, the following antibody clones were used: EGFR (BV711 anti-human EGFR, BioLegend, Cat #352919), HER2 (BV421 anti-human CD340, BioLegend, Cat #324420), IL13R2a (APC anti-human CD213a2, BioLegend, Cat #354405), GPC-3, EpCAM. For T cells, the following antibodies were used: APC-labeled anti-human CD3 Antibody, FITC labeled anti-human CD45RO Antibody, APC labeled anti-human CCR7 Antibody. For CAR expression analysis, the following antigen was used: FITC-Labeled Human GPC-3, HER2 Proteins. In brief, cells were washed with 1×PBS supplemented with 1% FBS (FACS buffer) and stained for 30 min in the dark at room temperature, followed by washing in FACS buffer before analysis.

### In Vivo Pharmacological Efficacy and Toxicology Studies

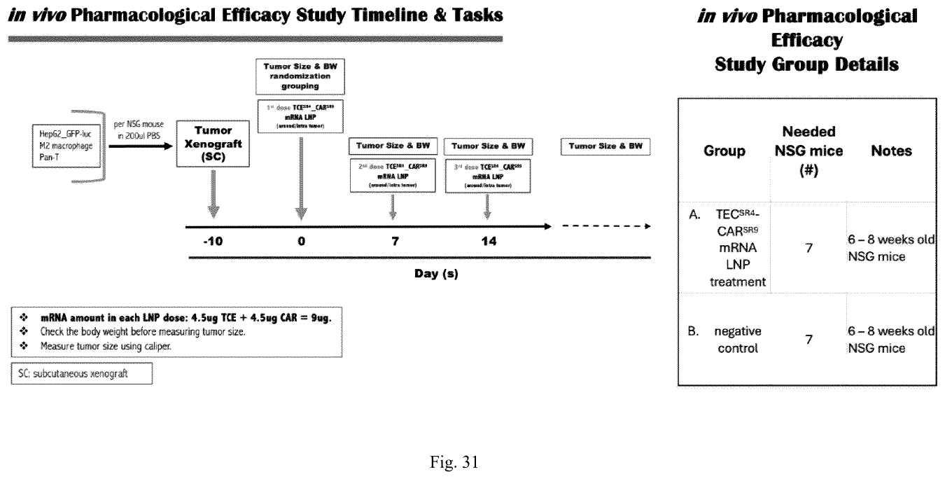

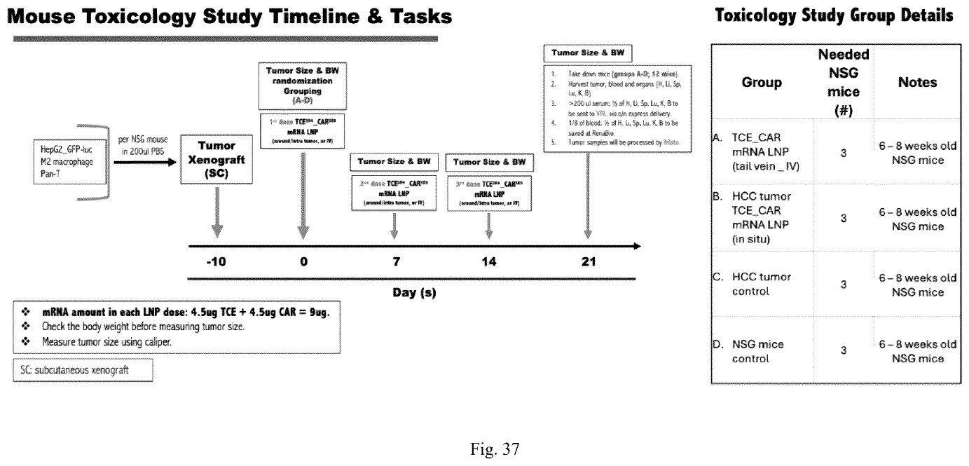

All animal studies were performed using NSG mice (NOD.scid IL2Rγcnull), which are a highly immunodeficient strain of laboratory mice lacking mature T cells, B cells, and Natural Killer (NK) cells. Studies were performed according to the details set forth below and as shown in FIGS. 31 and 37.

### Example 1: Polarization of Human Monocytes into M0 Macrophages

Cultured human monocytes were treated with phorbol myristate acetate (PMA) to polarize them into an M0 phenotype. M0 polarization was confirmed morphologically and by expression of CD68. The results are shown in FIG. 1.

### Example 2: Polarization of Human M0 Macrophages into MI Macrophages

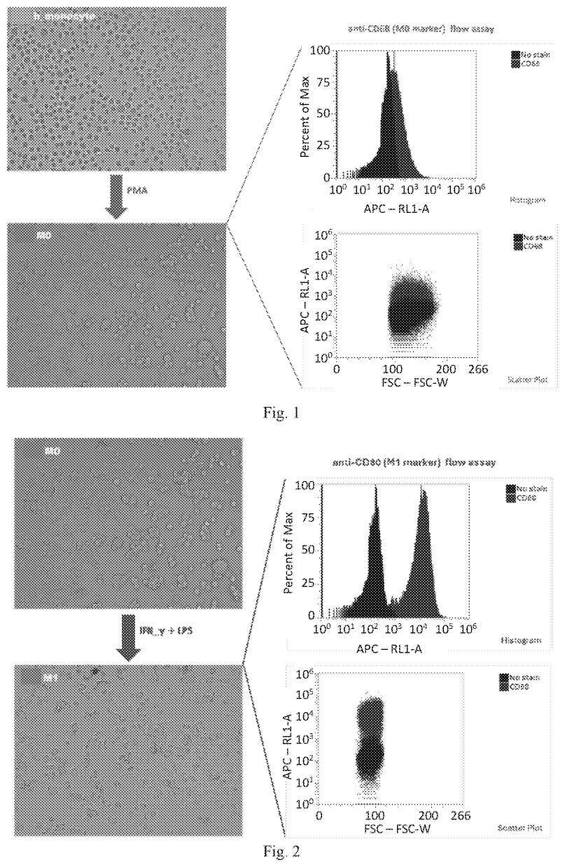

Human M0 macrophages were treated with interferon gamma (INF-γ) and lipopolysaccharide (LPS) to polarize them to an M1 phenotype. M1 polarization was confirmed morphologically and by expression of CD80. The results are shown in FIG. 2.

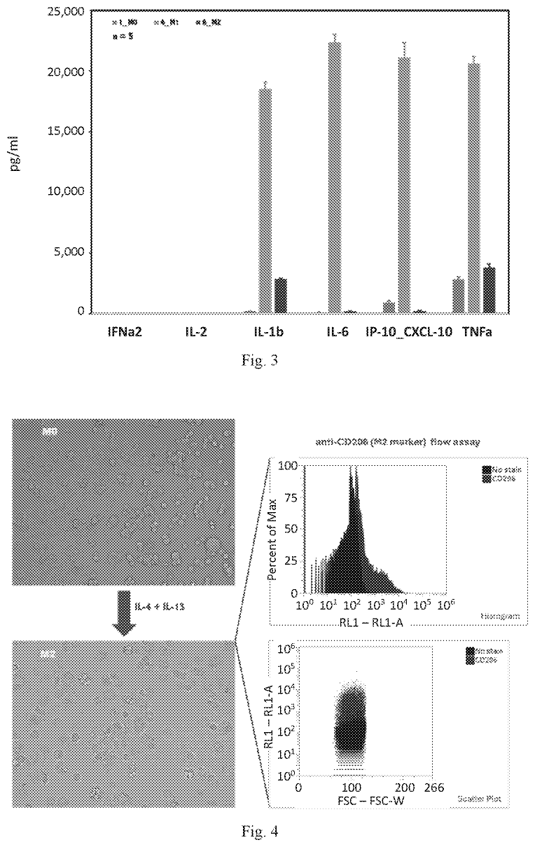

Culture medium from the M1 macrophages was collected and assayed for cytokine and chemokine secretion (IL-1β, IL-6, TNFα, and CXCL-10) using a Luminex assay. The results are shown in FIG. 3. The M1 macrophages secreted high levels of IL-1β, IL-6, TNFα, and CXCL-10 and did not secrete measurable amounts of INF-α2 or IL-2.

### Example 3: Polarization of Human M0 Macrophages into M2 Macrophages

Cultured M0 macrophages were treated with IL-4 and IL-13 to polarize them into an M2 phenotype. M2 polarization was confirmed morphologically and by expression of CD206. The results are show in FIG. 4.

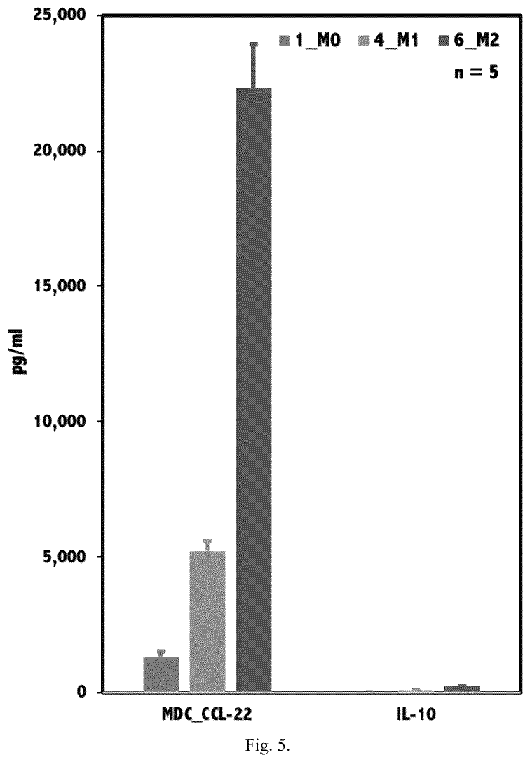

Culture medium from the M2 macrophages was collected and assayed for chemokine secretion (CCL-22) using a Luminex assay. The results are shown in FIG. 5. The M2 macrophages secreted CCL-22 (see FIG. 5) and did not secrete measurable amounts of INF-α2 or IL-2 (see FIG. 3).

### Example 4: Transfection of M2 Macrophages with LNP

Several LNPs were produced to examine their efficiency in transfecting M2 macrophages. The LNPs produced included an ionizable lipid, cholesterol, a phospholipid, and a pegylated lipid at a mole percent of 37% to 47%, 42% to 50%, 7% to 12%, and 1.5% to 5.5%, respectively. The core buffer was Na acetate at a pH of 4.5 to 6.5.

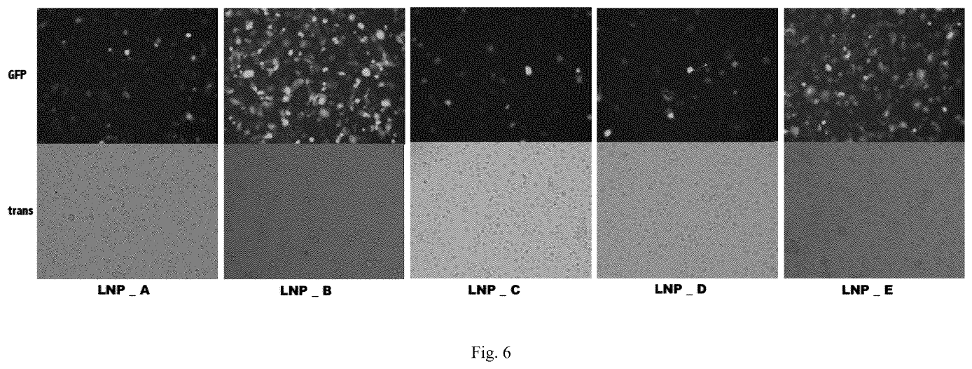

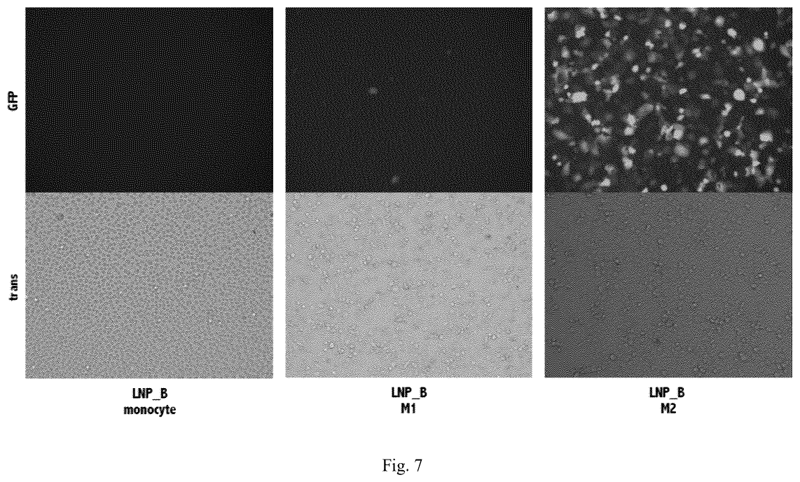

mRNA encoding green fluorescent protein (GFP) was prepared by in vitro transcription as follows. Plasmid DNA carrying the gene to be transcribed was linearized and in vitro transcription was performed using T7 polymerase and a cap under standard conditions. The resulting mRNA was purified and encapsulated in the LNPs at a molar ratio of the ionizable lipid to the mRNA of 3.3:1 to 5.6:1 and used to transfect M2 macrophages produced as described above. Exemplary LNPs are shown in Table 3 below and their transfection efficiency shown in Table 3 and in FIG. 6.

TABLE 3
LNP formulations

| | | Molar Ratio of | | |
|---|---|---|---|---|
| mRNA | | Ionizable Lipid | % of | |
| LNP | | to Nucleic | Transfected | Expression |
| Type | pegylated Lipid | Acids | Cells | Efficacy (+) |
| LNP_A | DMG-PEG-2000- | 4.5:1 | 25 | + |
| | Mannose | | | |
| LNP_B | DMG-PEG-2000 | 4.5:1 | 95 | ++++ |
| LNP_C | DSPE-PEG 2000 | 4.5:1 | 11 | + |
| LNP_D | DSPE-PEG 2000- | 4.5:1 | 12 | + |
| | Mannose | | | |
| LNP_E | 75% of DMG- | 3.5:1 | 95 | +++ |
| | PEG-2000 | | | |
| | 25% of DSPE- | | | |
| | PEG 2000- | | | |
| | Mannose | | | |

### Example 5: Selective Transfection of M2 Macrophages

The transfection efficiency of a nanoparticle of the invention was evaluated in human monocytes M1 macrophages, and M2 macrophages. GFP mRNA LNP_B of Example 4 was diluted into culture media and added to cultured monocytes, M1 macrophages, and M2 macrophages and expression of GFP was observed one day later by fluorescence microscopy. The results are shown in FIG. 7 and Table 4 below. The GFP mRNA-LNP of the invention selectively transfected human M2 macrophages with a very high efficiency of gene transduction (>95%) and a high level of expression efficacy.

TABLE 4
Selective transfection of M2 macrophages

| | % of transfected | Expression |
|---|---|---|
| Cell Type | cells | efficacy (+/−) |
| Monocyte | 0 | − |
| M1 | 1.5 | +/− |
| M2 | >95 | +++ |

% of transfected cells: average of 3 studies
expression efficacy: GFP fluorescence intensity of 3 studies

### Example 6: Long Term Gene Expression in Transfected M2 Macrophages

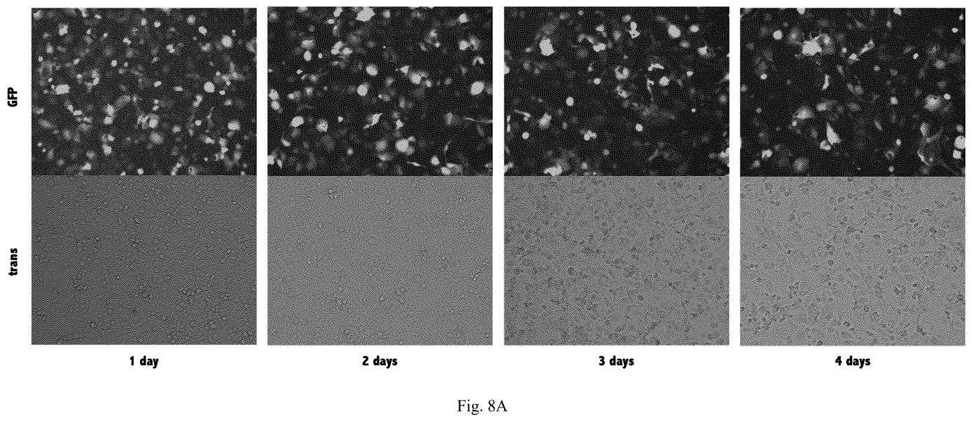

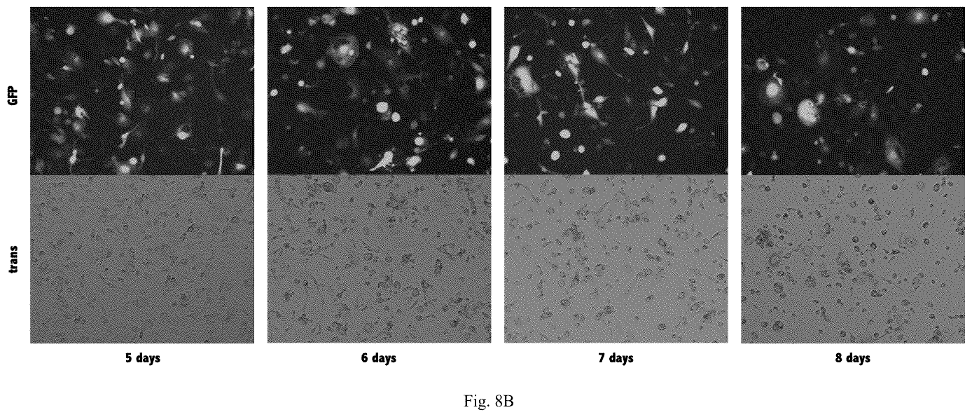

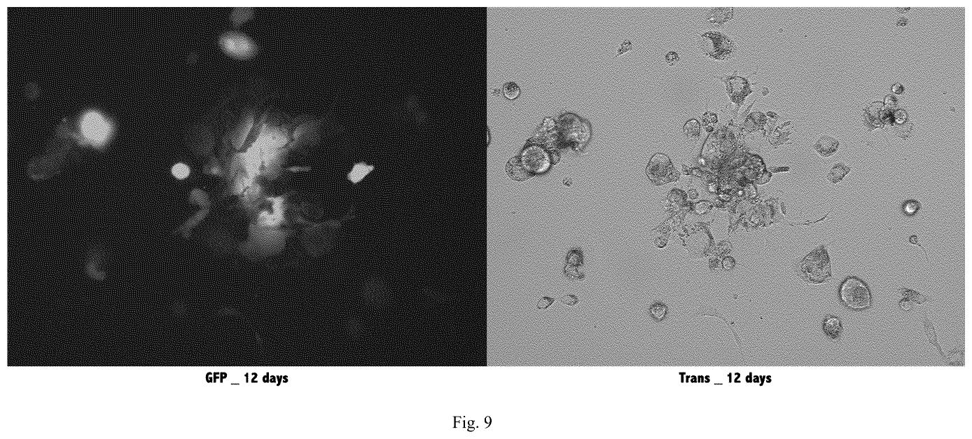

M2 macrophages were transfected as described above in Example 4. Robust persistent expression of GFP was seen in transfected cells for at least 12 days post-transfection. See FIGS. 8A, 8B, and 9. A GFP mRNA-LNP of the invention not only selectively transfects human M2 macrophage with very high efficacy of gene transduction and expression but also keeps robust persistent gene expression.

### Example 7: Expression of Multiple Genes in Transfected M2 Macrophages

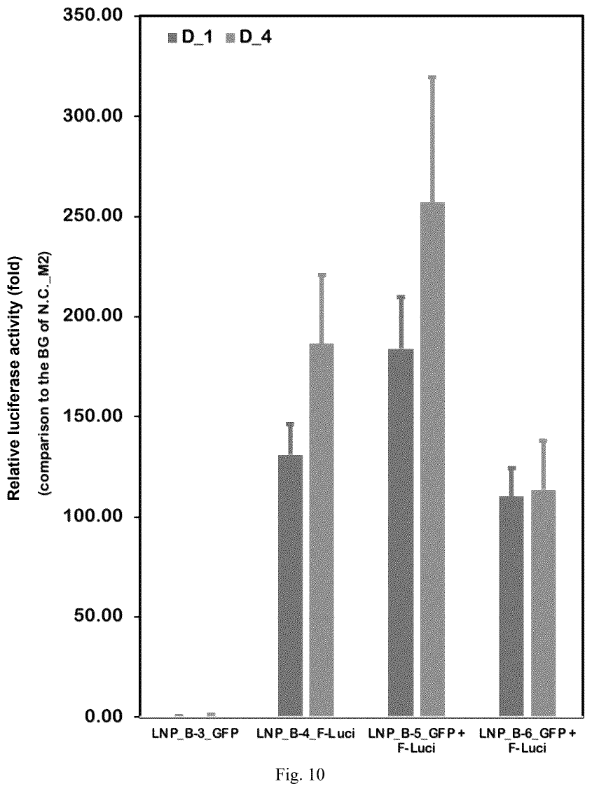

The transfection efficiency and gene expression level of two independent genes was tested after transfection into M2 macrophages. mRNAs for GFP and for firefly luciferase (F-Luci) were produced by in vitro transcription, encapsulated in a lipid nanoparticle of the invention singly or together, and transfected into M2 macrophages. The transfection efficiency and expression levels of GFP and F-Luci were measured by fluorescence microscopy and luciferase assay, respectively, at 1 and 4 days post-transfection. The results are shown in FIGS. 10 and 11.

As expected, M2 macrophages transfected with GFP mRNA-LNP had no detectable levels of luciferase activity, and M2 macrophages transfected with F-Luci mRNA-LNP had luciferase activity at least 130-fold over the background level of untransfected cells. Unexpectedly, M2 macrophages transfected with an LNP of the invention encapsulating both GFP mRNA and F-Luci mRNA had significantly higher luciferase activity, as compared to cells transfected with F-Luci mRNA alone. See FIG. 10.

A similar unexpected increase in GFP expression was seen in M2 macrophages transfected with GFP/F-Luci mRNA-LNP, which showed higher levels of transfection efficiency and GFP expression, as compared to cells transfected with GFP mRNA alone. See FIG. 11.

### Example 8: Transfection with an LNP of the Invention Stimulates the Polarization Transition from M2 to M1

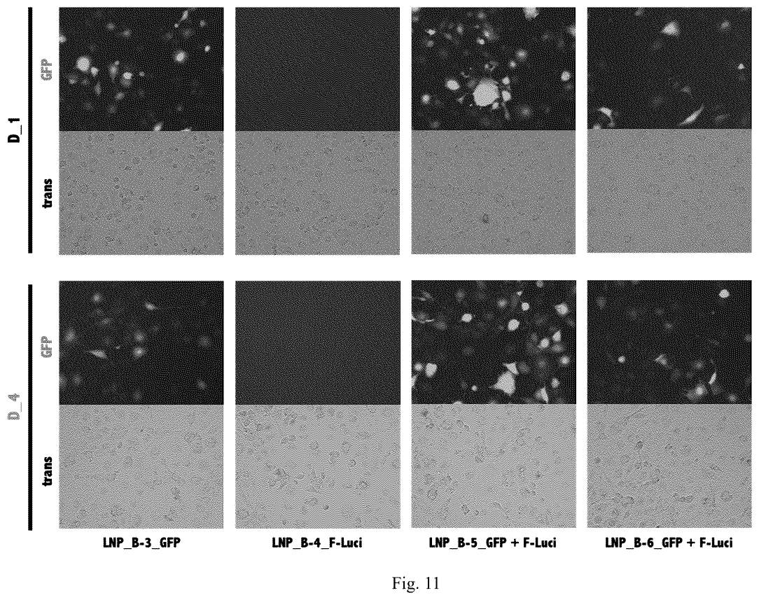

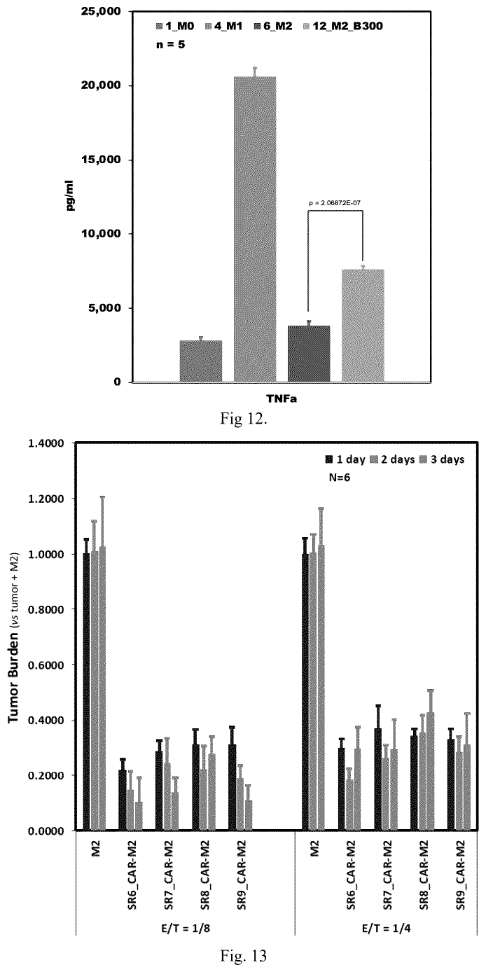

M2 macrophages were transfected with an LNP of the invention and TNF-α secretion into the culture medium was measured by a Luminex assay two days after transfection. The results are shown in FIG. 12.

Unexpectedly, M2 macrophages transfected by an LNP of the invention secreted at least 100% more TNFα, as compared to untransfected M2 macrophages. See FIG. 12. For reference, the levels of TNF-α production of untransfected M0, M1, and M2 macrophages are also shown. This data shows that the LNP of the invention can stimulate the polarization transition from an M2 anti-inflammatory phenotype towards an M1 pro-inflammatory phenotype.

### Example 9: Identification of HCC Lead CAR mRNA LNP with High Potency at a Low E/T Ratio

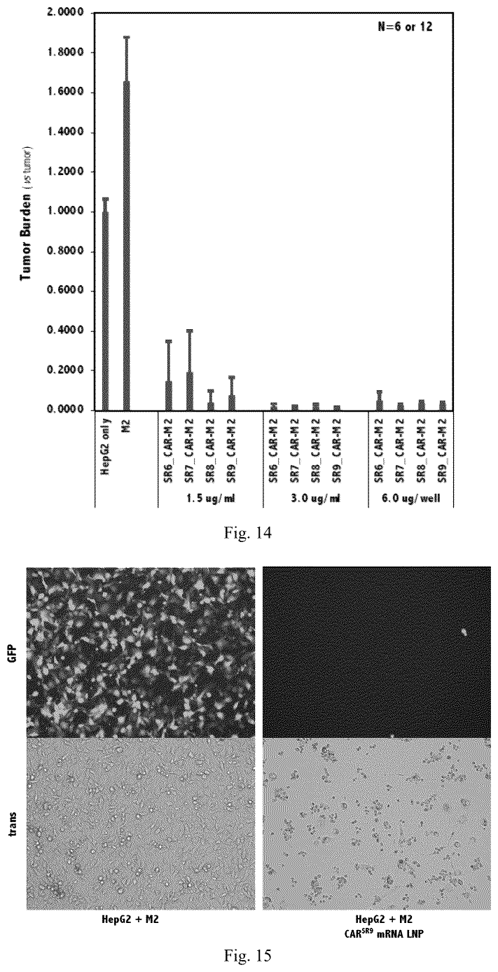

Tumor-associated macrophages (TAM; M2) are a dominant population of immune cells in the HCC TME and key contributors to TME immune suppression. Targeting TAM and flipping their immune suppression switch is an ideal therapeutic approach. A series of anti-glypican 3 (GPC3) CARs were constructed with differentiated intracellular stimulation/activation domains (constructs CAR_SR6-SR9; see Table 2 above). mRNA for each construct was prepared by in vitro transcription and encapsulated into LNP as described above to produce CAR mRNA LNPs. HepG2 HCC cells stably expressing both GFP and luciferase (HepG2_GFP/Luci) were co-cultured with M2 macrophages produced as described above at different effector (M2) to target (HCC) ratios and treated with CAR mRNA LNPs. Luciferase activities in the cocultures were measured at different times after CAR mRNA LNP treatment and compared to cocultures not treated with the CAR mRNA LNPs. The results are shown in FIGS. 13 and 14. CAR_SR9 was selected as the most potent anti-GPC-3 CAR at the lowest E/T ratio of 1/8.

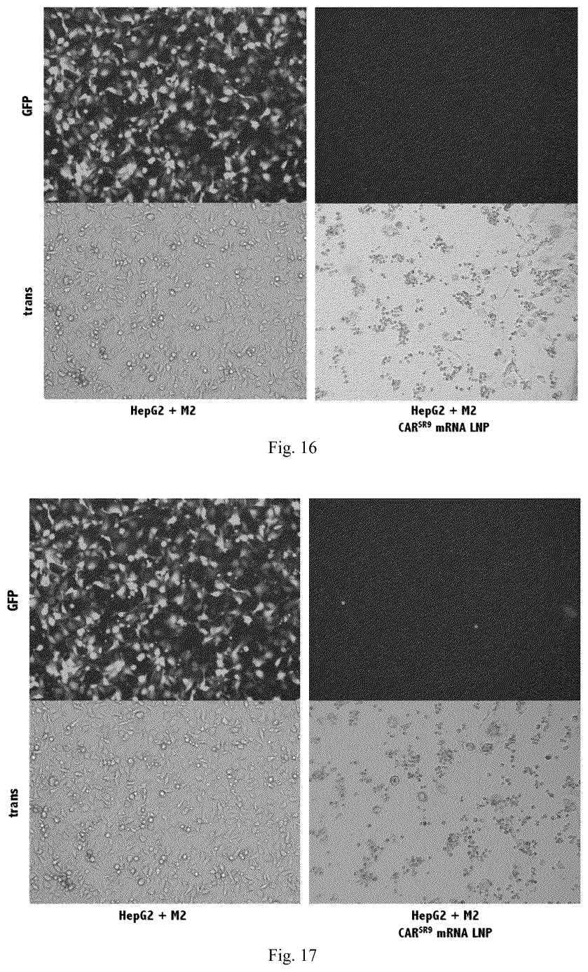

The effectiveness of CAR_SR9 was confirmed by directly observing GFP expression in the HCC cells cocultured with M2 and treated with the CAR mRNA LNP. The results are shown in FIGS. 15-17.

### Example 10: Identification of HCC Lead TCE mRNA LNP with High Potency at a Low E/T Ratio

To further harness the therapeutic potentials of LNPs that selectively target human M2 macrophages, a series of anti-EGFR and/or -EpCAM T Cell Engager (TCE) constructs were made (constructs TCE_SR1-SR5; see Table 2 above) and used as IVT templates to produce mRNA, which was encapsulated into LNPs. The resulting TCE mRNA LNP were used to transfect M2 macrophages that subsequently secreted the TCEs.

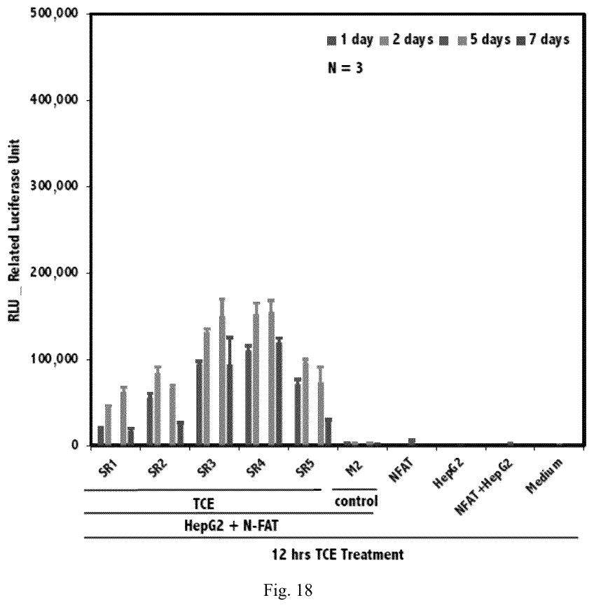

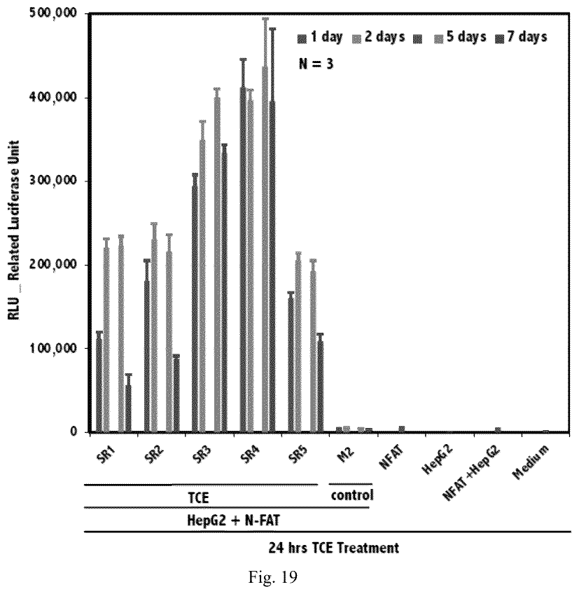

Supernatants from transfected cultured M2 macrophages were used in a T cell activation assay that employed cocultures of HepG2 cells and Jurkat T cells stably transfected with an NFAT-luciferase construct as described above. The results are shown in FIGS. 18 and 19. The TCE secreted into transfected M2 culture supernatants activated the Jurkat T cells in a manner dependent on the presence of HepG2 cells.

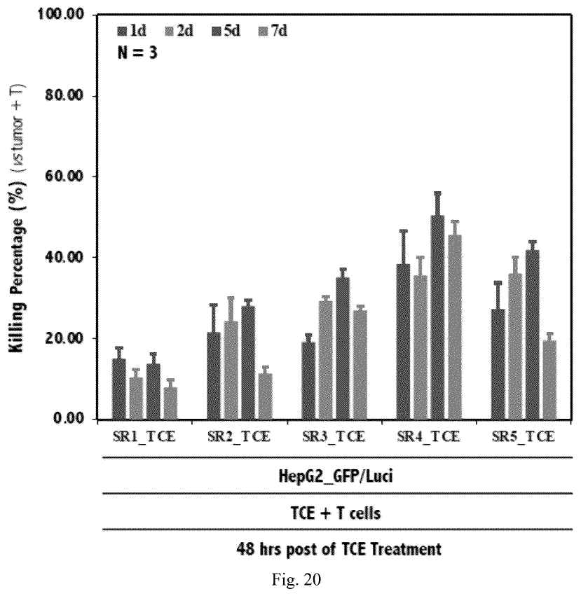

The ability of the secreted TCE to activate T cells to kill HCC cells was tested by treating cultured HepG2_GFP/Luci cells with Pan-T cells and the secreted TCE. The results are shown in FIGS. 20 and 21. All of the TCE induced T cells to kill the HCC cells to a degree, with the most potent being TCE_SR4.

### Example 11: Identification of HCC Lead Composition of TCE_CAR mRNA LNP with High Potency Even at an Extremely Low E/T Ratio

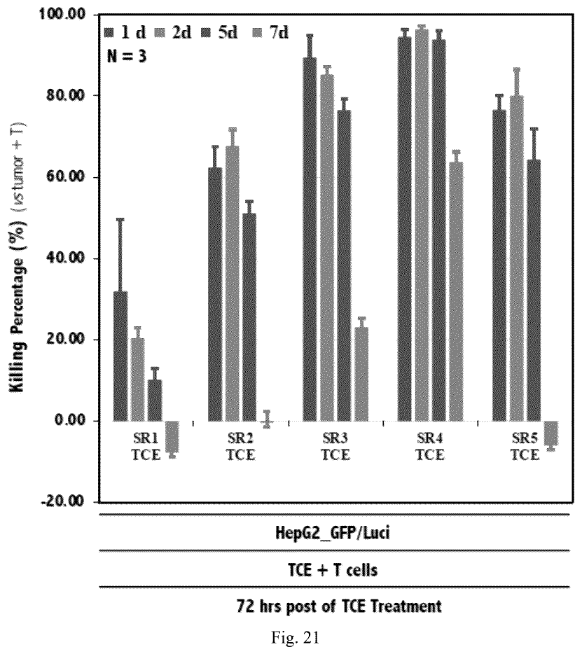

After identification of lead CAR and TCE for treating HCC, both CAR and TCE mRNAs were simultaneously encapsulated into LNP targeting M2 macrophages. The resulting TCE_CAR mRNA LNP (more specifically TCE^{SR4}_CAR^{SR9} mRNA LNP) was tested for its ability to kill HCC cells (HepG2_GFP/Luci) cocultured with M2 macrophages and T cells. The results are shown in FIGS. 22-25.

Treatment of HCC/M2/T cell cocultures with TCE_CAR LNP resulted in significantly more killing of the HCC cells, as compared to CAR mRNA LNP, despite the fact that the TCE_CAR LNP carries half the amount of mRNA for the CAR compared to the CAR mRNA LNP. See FIG. 22.

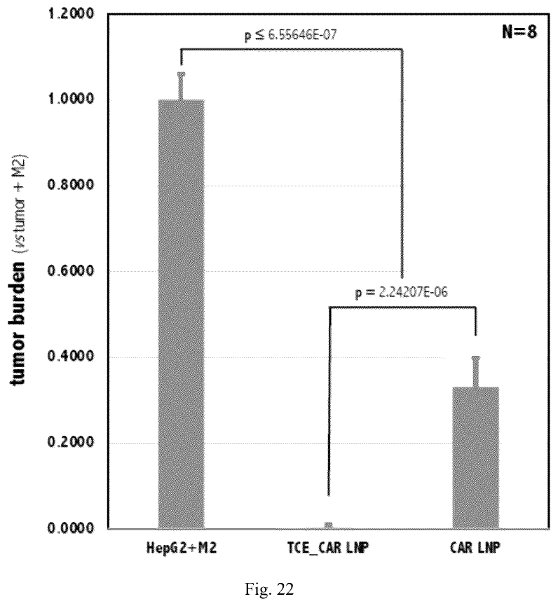

A similar study showed that the potency of the TCE_CAR LNP for killing HCC was significantly better than the TCE mRNA LNP. See FIG. 23.

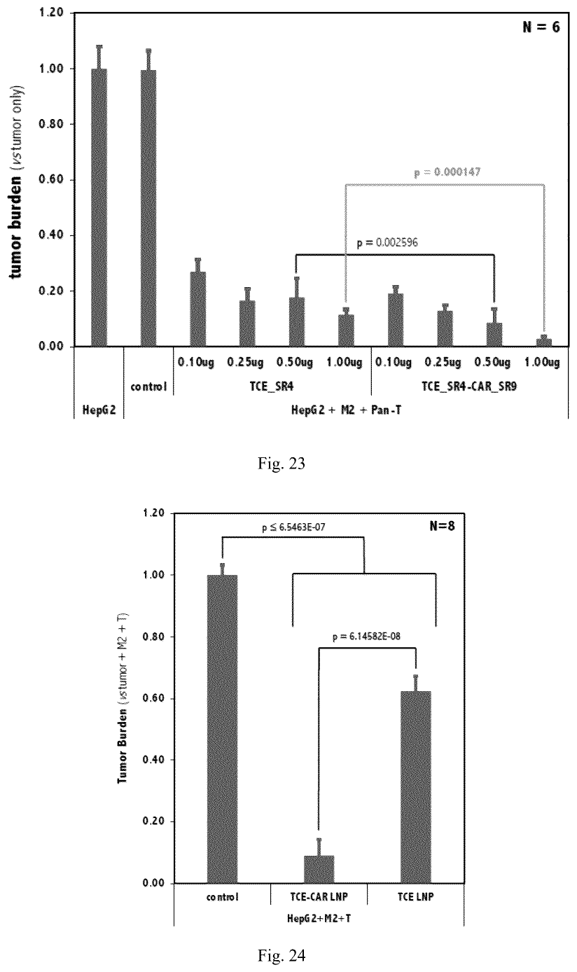

Further studies revealed that, even at very low E/T ratios (E/T M2=1/64; E/T T=1/32), the TCE_CAR mRNA LNP killed significantly more HCC cells compared to the TCE mRNA LNP. See FIG. 24.

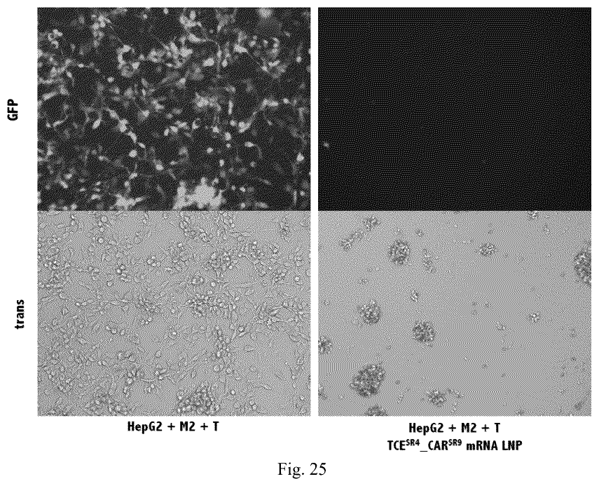

The effectiveness of the TCE_CAR mRNA LNP was confirmed by directly observing GFP expression in the HCC cells cocultured with M2 and T cells and treated with TCE^{SR4}_CAR^{SR9} mRNA LNP. See FIG. 25.

### Example 12: Repeat Dosing of the Lead TCE_CAR mRNA LNP has a Robust Killing Potency to Human HCC Cancer Cells

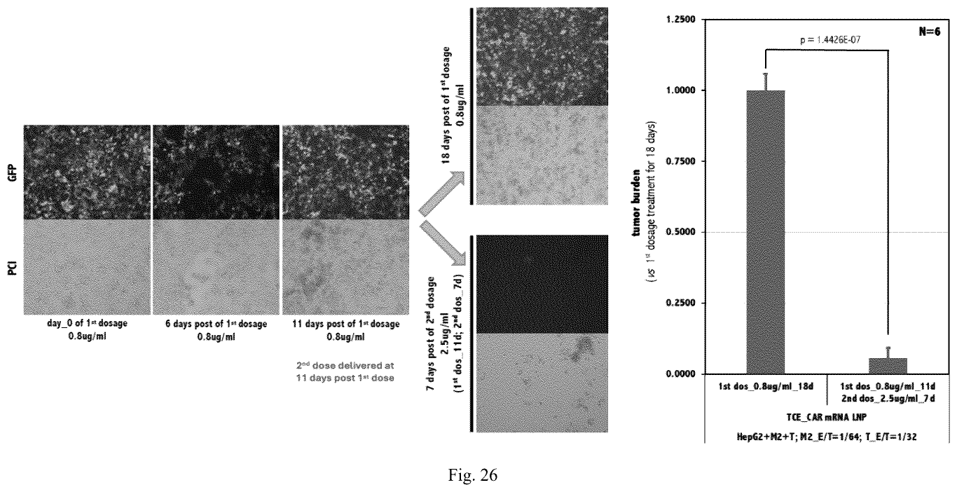

An in vitro co-culture study was performed to directly address the question whether recurrent HCC tumor can be eradicated by repeated dosing of TCE_CAR mRNA LNP. The results are shown in FIG. 26. HepG2_GFP/Luci cells were co-cultured with M2 macrophages and T cells and treated with 0.8 μg/mL TCE^{SR4}_CAR^{SR9} mRNA LNP. Significant killing of the HCC cells was seen after 6 days, but HCC cell numbers returned to pre-treatment levels by 11 days post-treatment. See FIG. 26, left panel. On day 11, some of the previously treated HCC cells received a second dose of TCE^{SR4}_CAR^{SR9} mRNA LNP and others were left untreated. The results showed that nearly all of the HCC cells receiving the second dose were killed within 7 days (18 days after the initial treatment) as compared to cells treated only once. See FIG. 26, center and right panels. This repeat dose study indicated that TCE^{SR4}_CAR^{SR9} mRNA LNP had a robust killing activity against recurrent human HCC cancer cells.

### Example 13: Generation of Memory T Cells (Cancer Vaccine) Via the Interplay of CAR and TCE

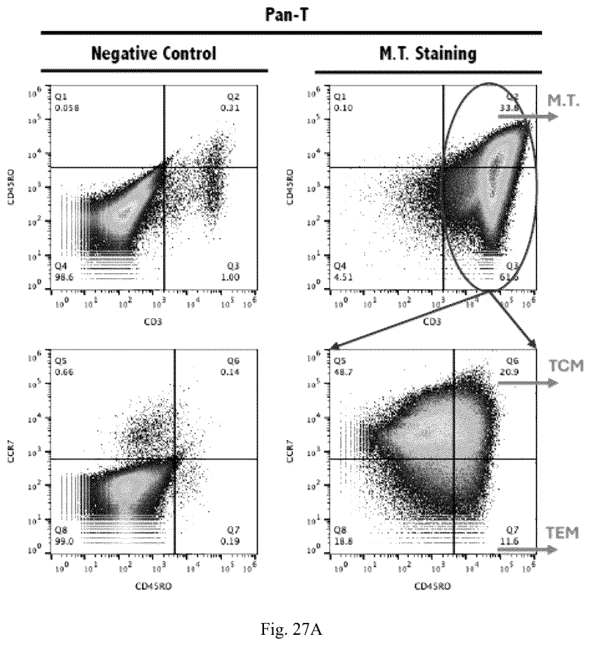

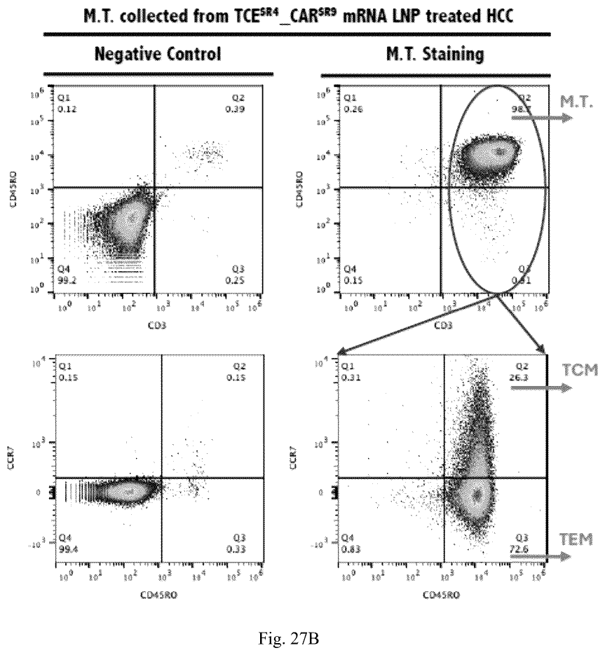

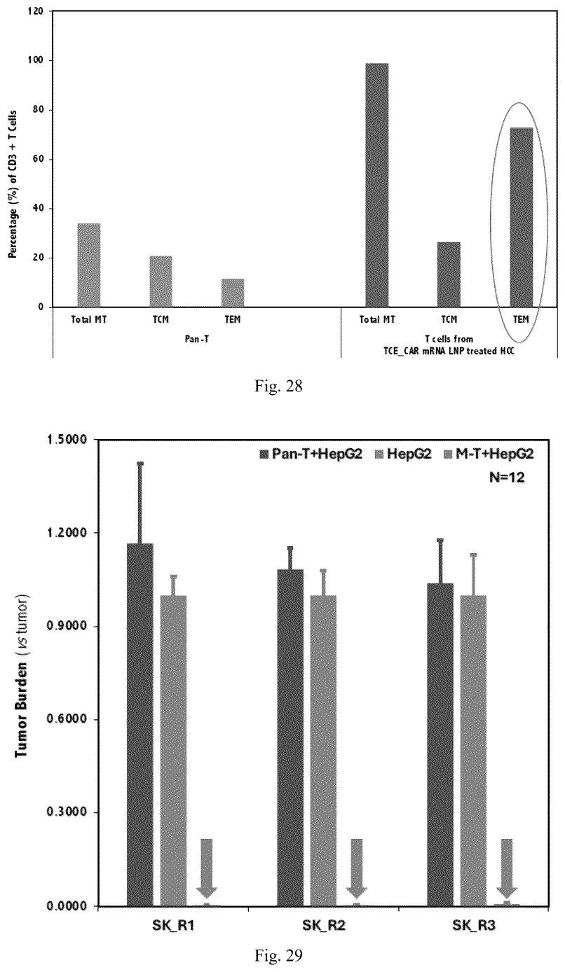

The effect of the above treatments on T cell populations was examined by flow cytometry analysis. As shown in FIGS. 27A and 28, about 34% of Pan-T cells were memory T cells (MT), with 21% being central memory T cells (TCM) and 12% being effector memory T cells (TEM).

The T cells that were expanded during the HCC cell-killing study described in Example 11 and shown in FIG. 25 (TCE^{SR4}_CAR^{SR9} mRNA LNP treated HCC cancer cells co-cultured with M2 macrophages and T cells) surprisingly were 99% MT, with 26% TCM and 73% TEM. See FIGS. 27B and 28.

The MT cells recovered from the HCC cell-killing study effectively killed HCC cells in the absence of added M2 cells. See FIG. 29, SK_R1. T cells recovered from SK_R1 were similarly effective at killing HCC cells. See FIG. 29, SK_R2. Another serial round of killing showed again that T cells from the second serial killing round were effective for killing HCC cells. See FIG. 29, SK_R3.

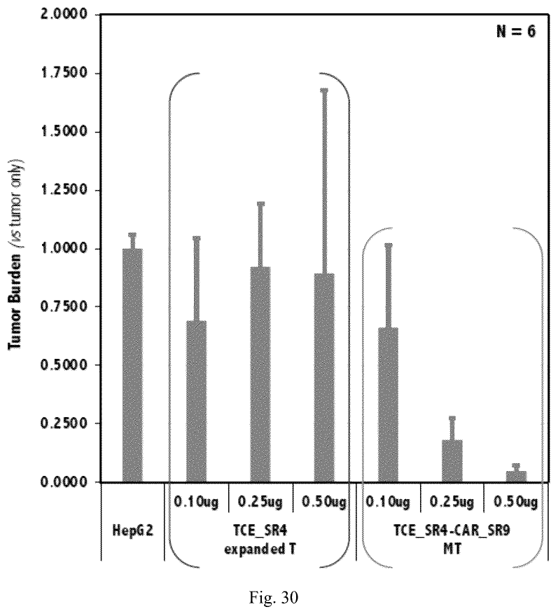

T cells expanded from the coculture studies described in Example 11 and shown in FIG. 23 were tested for their ability to kill HCC cells. The results are shown in FIG. 30. Surprisingly, T cells from cocultures of HCC/M2/T cells treated with TCE^{SR4} mRNA LNP were relatively ineffective at killing HCC cells, while T cells from cocultures of HCC/M2/T cells treated with TCE^{SR4}_CAR^{SR9} mRNA LNP had significant ability to kill the HCC cells. This result indicates that the cancer vaccine response was generated via the interplay of CAR and TCE.

### Example 14: The Lead TCE_CAR mRNA LNP has a Significant In Vivo Therapeutic Efficacy Against HCC Tumor

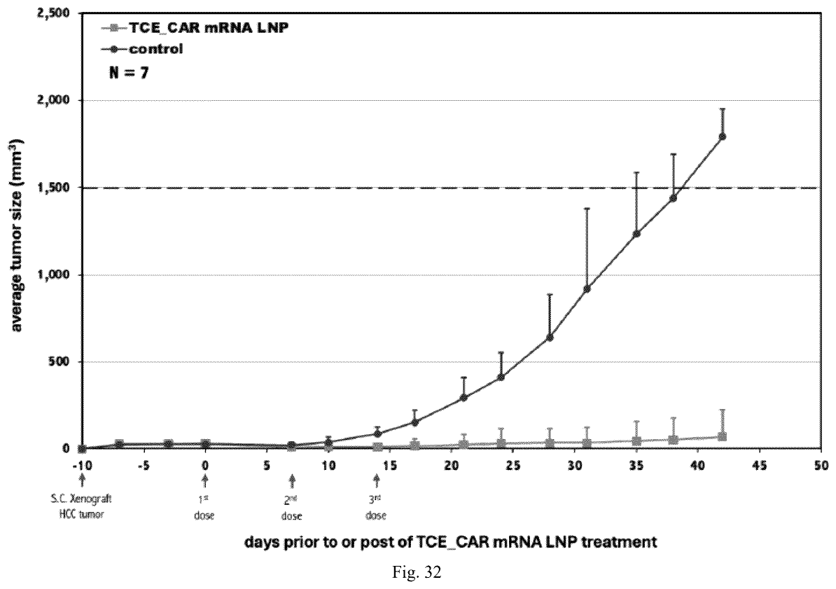

In vivo efficacy studies were performed in a murine model for HCC according to the design shown in FIG. 31. Briefly, HCC tumors were established in NSG mice by subcutaneous injection of 7.5×10⁶ HepG2_GFP/Luci cells, 2.5×10⁶ human M2 macrophages, and 2.5×10⁶ human T cells. Ten days later (Day 0) the mice were randomized to treatment group and control group. The treatment group mice were injected into or near the established tumors with an amount of TCE^{SR4}-CAR^{SR9} mRNA LNP containing 4.5 μg of each mRNA for a total of 9 μg. Control animals were injected with vehicle. Injections were repeated on day 7 and day 14, and tumor size measured on days −7, −3, 0, 7, 10, 14, 17, 21, 24, 28, 31, 35, 38, and 42. The tumor size measurements are shown in FIG. 32. Treatment with TCE^{SR4}-CAR^{SR9} mRNA LNP significantly reduced tumor size by day 14. See Table 5 below.

TABLE 5
Significant differences in tumor size between
TCE^{SR4}-CAR^{SR9} mRNA LNP treated and controls

| Days | 0 | 7 | 10 | 14 | 17 | 21 |
|---|---|---|---|---|---|---|
| p | 0.06322799 | 0.13246355 | 0.06077777 | 0.0069429 | 0.00343986 | 0.00137466 |
| Days | 24 | 28 | 31 | 35 | 38 | 42 |
| p | 0.00080167 | 0.0054513 | 0.00107546 | 7.0924E−05 | 6.8163E−06 | 6.3335E−07 |

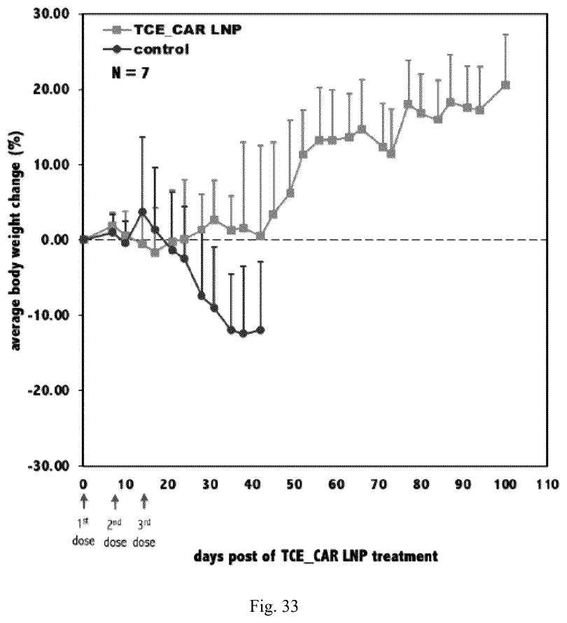

Average body weight of treated and control mice were also measured starting on day 0. The results are shown in FIG. 33. Control mice lost a significant amount of body weight over the course of the study, while treated mice maintained body weight and then showed an increase after day 42. The increase in weight was expected for the age of the mice.

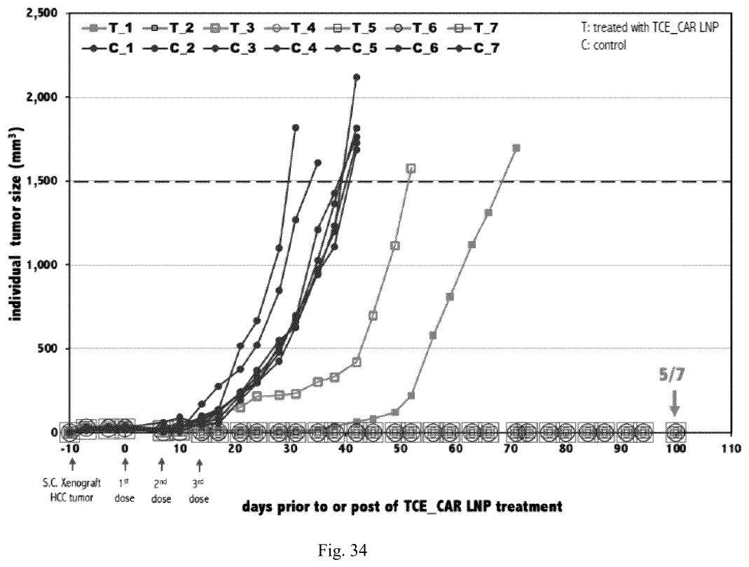

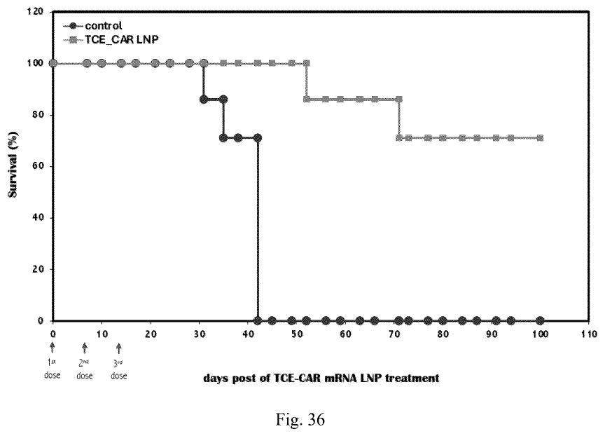

Tumor size measurements from individual animals are shown in FIG. 34. Remarkably, 71% (5/7) of animals treated with TCE^{SR}4_CAR^{SR9} mRNA LNP showed no tumor growth or a reduction in tumor size, i.e., cancer-free, up to 100 days after the first dose. See also FIG. 36.

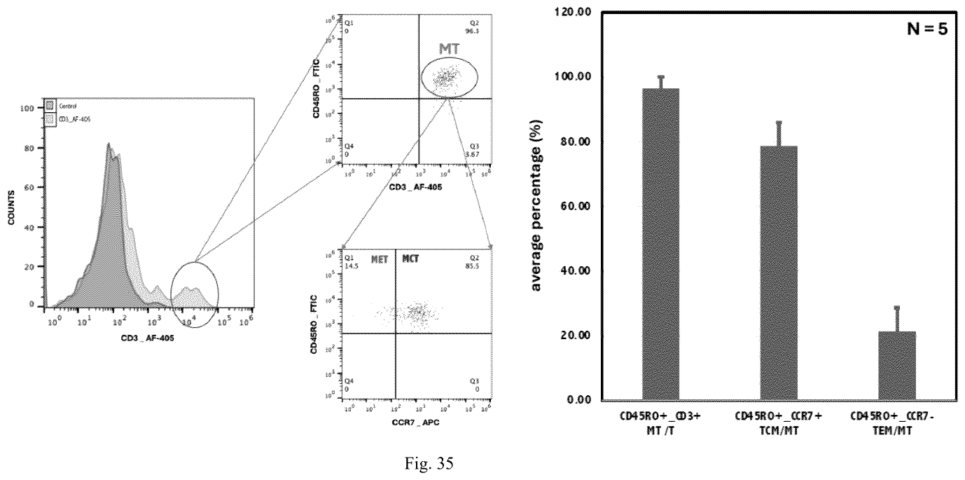

The spleen cells collected from the cancer-free mice (FIG. 34) were analyzed using flow cytometry assay. The results are shown in FIG. 35. The majority (>90%) of human T cells isolated from the murine spleens were MT cells, with ~80% being TCM. This result strongly suggests that these memory T cells contribute to the long-term cancer-free survival of the TCE^{SR4}_CAR^{SR9} mRNA LNP treated animals.

### Example 15: The Lead TCE_CAR mRNA LNP has No Detectable Toxicity

In vivo toxicology studies were performed in the murine model for HCC described in Example 14 according to the design shown in FIG. 37. The study was essentially identical to the in vivo efficacy study except that animals were randomized to 4 groups as shown in FIG. 37, and the study was terminated after 21 days post-treatment.

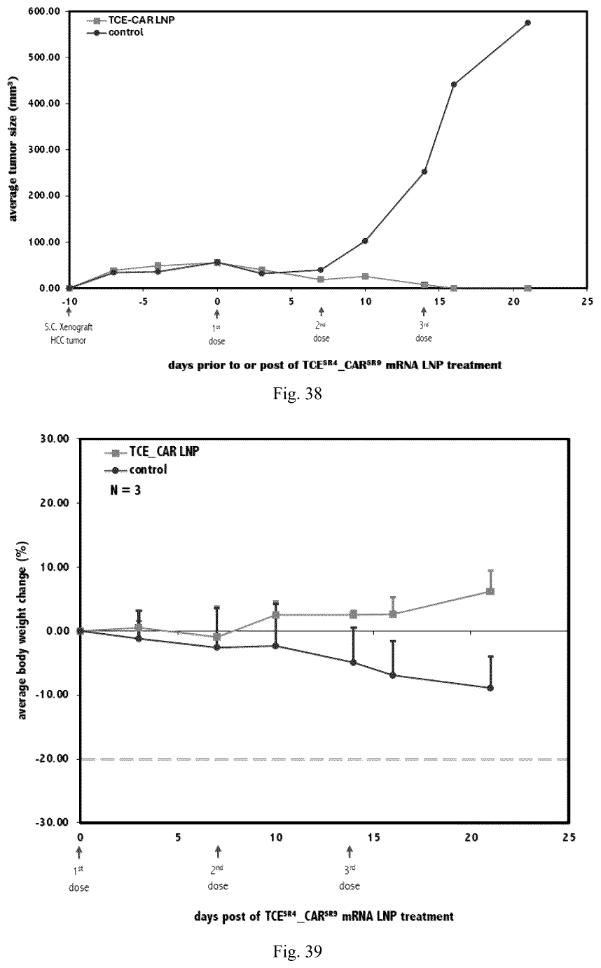

Measurements of average tumor size (FIG. 38) and body weight (FIG. 39) confirmed the results from the efficacy study described in Example 14.

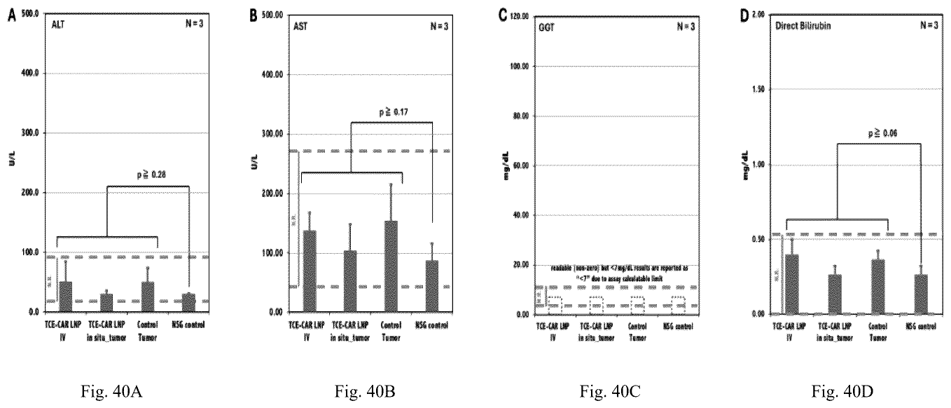

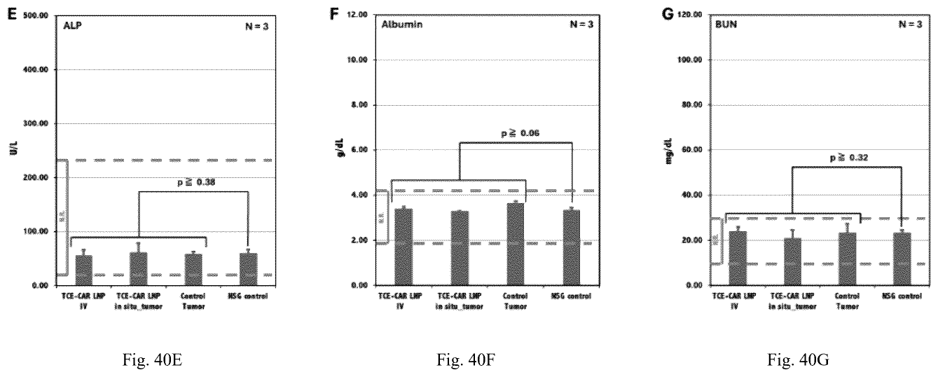

Toxicity of the TCE^{SR4}_CAR^{SR9} mRNA LNP treatment was assessed by examining the serum levels of liver and kidney biomarkers. The results are shown in FIGS. 40A-40G. No liver or kidney toxicity was detected in animals injected intravenously or around the tumor with the TCE^{SR4}_CAR^{SR9} mRNA LNP.

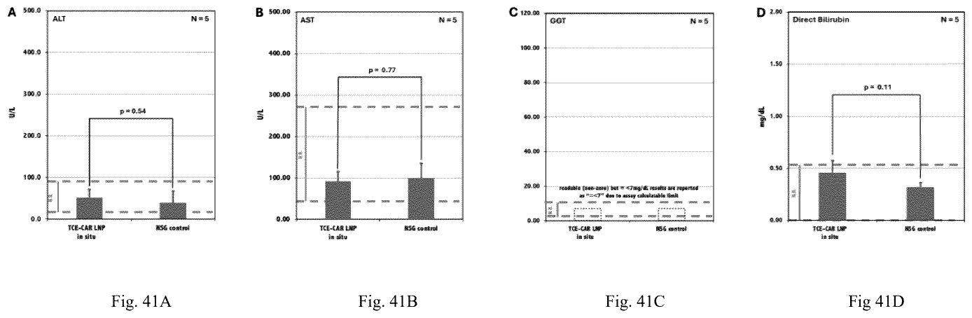

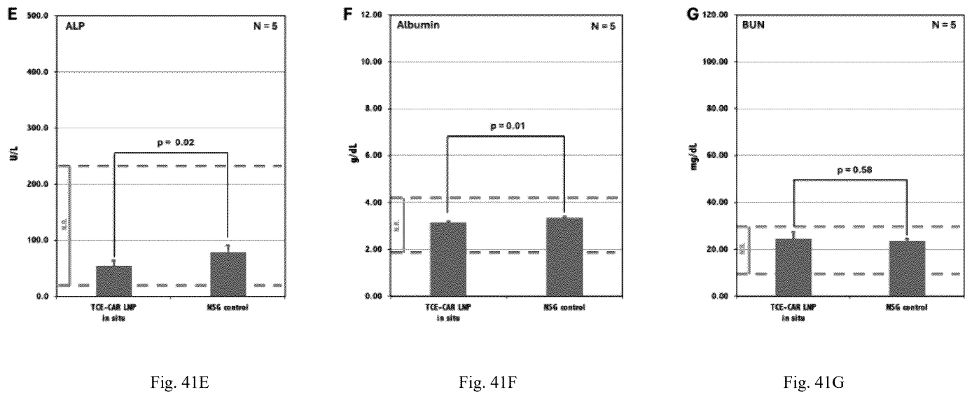

Serum samples from the cancer-free animals described in Example 14 were also analyzed for liver and kidney biomarkers. The results, shown in FIGS. 41A-41G, also show no toxicity of the TCE^{SR4}_CAR^{SR9} mRNA LNP treatment.

Patho-histological examination of major organs from the experimental animals confirmed that there were no major adverse effects of the TCE^{SR4}_CAR^{SR9} mRNA LNP treatment. The results are summarized below in Table 6 (in vivo toxicology study of Example 15) and Table 7 (TCE^{SR4}_CAR^{SR9} mRNA LNP treated, HCC cancer-free mice of Example 14).

TABLE 6
Histologic scoring of organs from experimental animals

| | | | | RNB-001 | | | | | | | | |
|---|---|---|---|---|---|---|---|---|---|---|---|---|
| | | | | HCC tumor | | | HCC tumor | | | | | |
| | RNB-001 (IV) | | | (in situ) | | | control | | | Normal NSG | | |
| Group | #1 | #2 | #3 | #1 | #2 | #3 | #1 | #2 | #3 | #1 | #2 | #3 |
| Heart | N | N | N | N | N | N | N | N | N | N | N | N |
| Liver | N | N | N | N/2MF | N/2MF | N | 3D | N | 3D | N | N | N |
| Spleen | N | N | N | N | N | N | N | N | N | N | N | N |
| Lung | N | N | N | 3D | 4D | N/2MF | 4D | N | 4D | N | N | N |
| Kidney | N | N | N | N | N | N | 3D | N | N/2MF | N | N | N |
| Brain | N | N | N | N | N | N | N | N | N | N | N | N |

2 = Mild
3 = Moderate
4 = Marked
N = Normal
MF = Multifocal
D = Diffuse

TABLE 7
Histologic scoring of organs from cancer-free animals

| | RNB-001 HCC tumor (in situ) | | | | | Normal NSG (control) | | | | |
|---|---|---|---|---|---|---|---|---|---|---|
| Group | #1 | #2 | #3 | #4 | #5 | #1 | #2 | #3 | #4 | #5 |
| Heart | N | N | N | N | N | N | N | N | N | N |
| Liver | N | N | N | N | N | N | N | N | N | N |
| Spleen | N | N | N/3F | N | N | N | N | N | N | N |
| Lung | N/2MF | N/2F | N | N | N | N | N | N | N | N |
| Kidney | N/1MF | N | N | N | N | N | N | N | N | N |
| Brain | N | N | N | N | N | N | N | N | N | N |

1 = Minimal
2 = Mild
3 = Moderate
N = Normal
MF = Multifocal
F = Focal

The observation of nodules of human lymphocytes and macrophages was as expected, which resulted from injection of human immune cells circulating in the NSG mice. Pathologies observed were: Liver, small nodules of lymphocytes and macrophages in perivascular connective tissue, primarily adjacent to central veins; Lung, small nodules of lymphocytes, cell debris and macrophages surrounding blood vessels, generally arterioles; Kidney, small nodules of lymphocytes, cell debris and macrophages throughout the cortex, corticomedullary area, medulla, and suburothelial pelvis.

### Example 16: Identification of GBM Lead TCE mRNA LNP with High Potency at a Low E/T Ratio

In addition to TAM-induced immune suppression in GBM, genetic heterogeneity is another key challenge to develop effective therapy for GBM. Tumor-associated antigen (TAA) expression was determined by flow cytometry in several cultured GBM cell lines. The results are shown in Table 8 below.

TABLE 8
Expression of GBM TAA in GBM cell lines

| | |
|---|---|
| GBM cell | GBM Tumor-Associated Antigen |

| line | EGFR (%) | HER2 (%) | IL13Rα2 (%) |
|---|---|---|---|
| U138 | 99.40 | 0 | 4.61 |
| U87 | 99.50 | 0.48 | 37.78 |
| U251 | 99.90 | 87.60 | 96.10 |

A series of anti-IL13Ra2 and/or EGFR TCEs were constructed based on the TAA expression data (TCE_SR28-212; see Table 2 above). mRNA for each construct was prepared by in vitro transcription and encapsulated into LNP as described above to produce TCE mRNA LNPs.

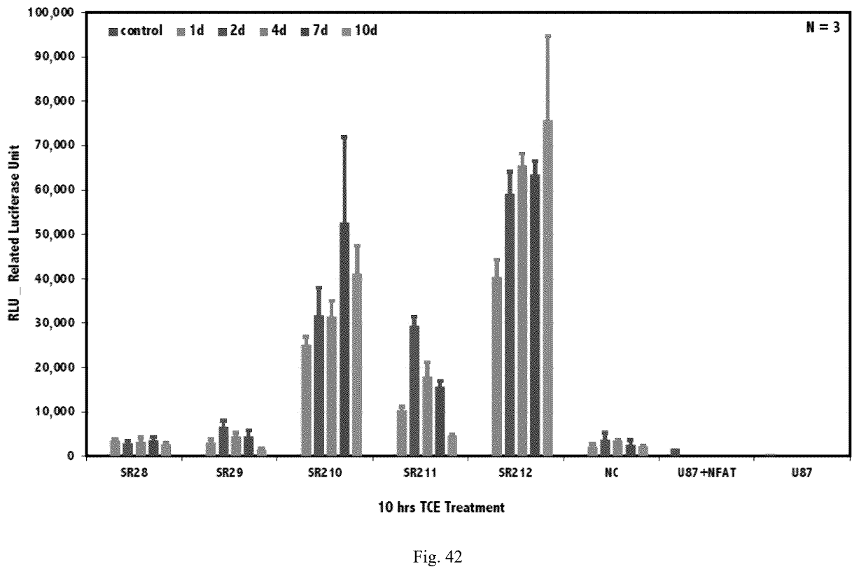

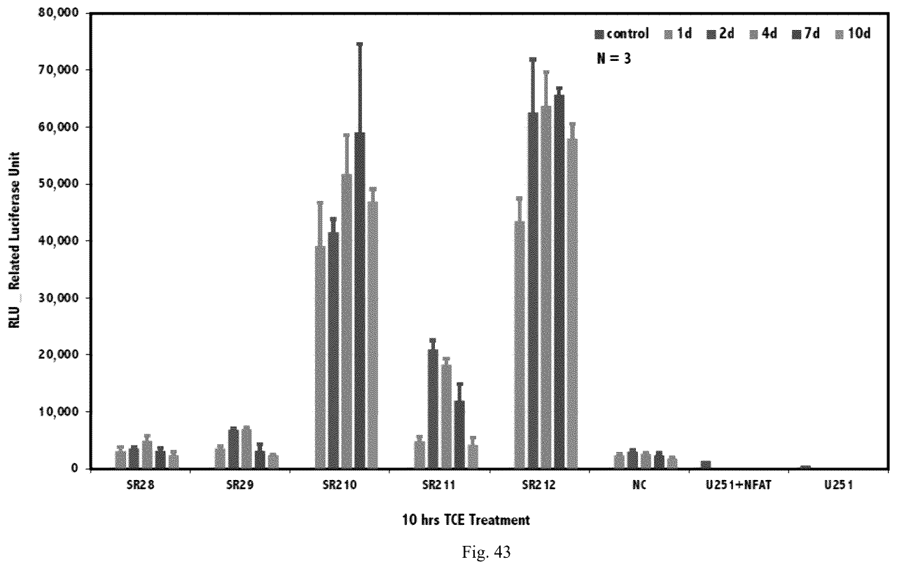

The TCE mRNA LNPs were first tested for their ability to activate T cells in the presence of GBM cells using the Jurkat NFAT-luciferase coculture system described above in Example 10. The results are shown in FIG. 42 for U87 cells and in FIG. 43 for U251 cells. The secreted TCE from TCE_SR²¹² mRNA LNP transfected M2 macrophages was the most efficient activator of T cells for both cell lines.

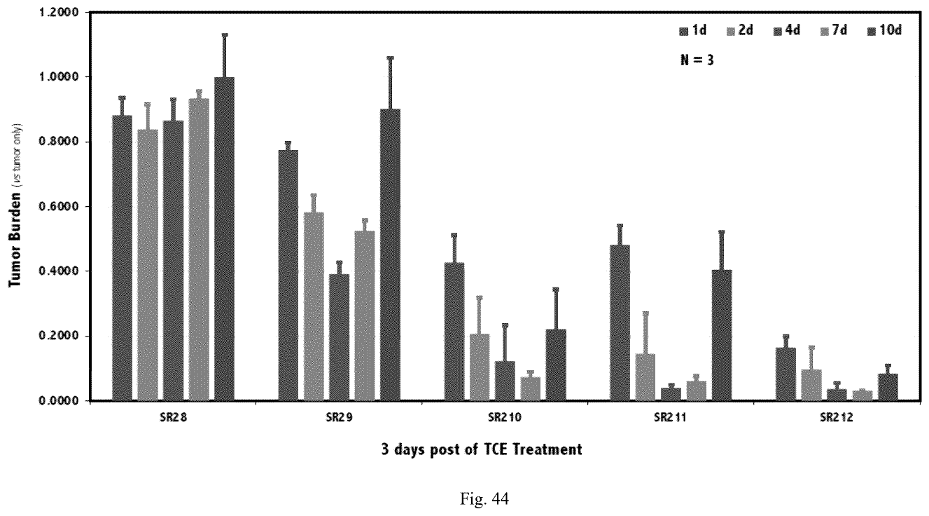

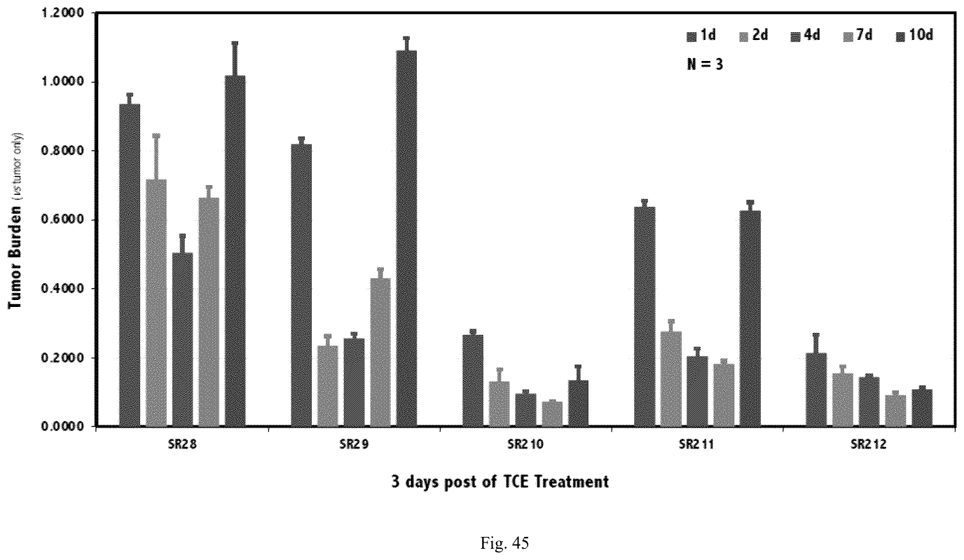

The ability of the secreted TCE to activate T cells to kill GBM cells was tested by treating cultured U138_GFP/Luci cells and U251 GFP/Luci cells with Pan-T cells and the secreted TCE. The results are shown in FIGS. 44 (U138) and 45 (U251). The results confirmed that TCE_SR²¹² was the most effective for killing GBM cells in the presence of T cells.

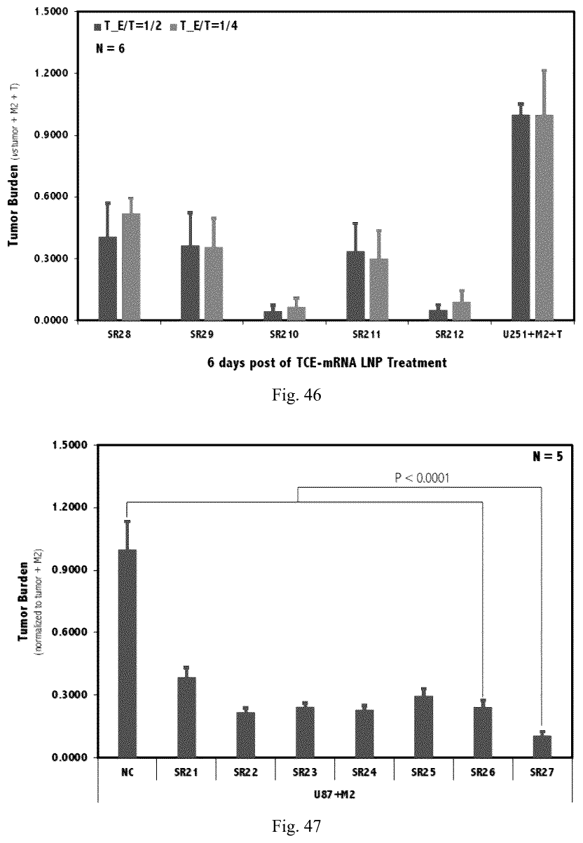

U251_GFP/Luci cells were cocultured with M2 macrophages and T cells and treated with the TCE mRNA LNP constructs described above. The results (see FIG. 46) showed that TCE_SR²¹⁰ and TCE_SR²¹² were the most effective for killing the GBM cells.

### Example 17: Identification of GBM Lead CAR mRNA LNP with High Potency at a Low E/T Ratio

A series of anti-HER2 and/or EGFR CARs were constructed with differentiated intracellular stimulation/activation domains (constructs CAR_SR21-SR27; see Table 2 above). mRNA for each construct was prepared by in vitro transcription and encapsulated into LNP as described above to produce CAR mRNA LNPs.

U251 GBM cells stably expressing both GFP and luciferase (U87_GFP/Luci) were co-cultured with M2 macrophages produced as described above at an effector (M2) to target (GBM) ratio of 1/8 and treated with the CAR mRNA LNPs. Luciferase activities in the cocultures were measured 3 days after CAR mRNA LNP treatment and compared to luciferase in cocultures not treated with the CAR mRNA LNPs. The results are shown in FIG. 47. CAR_SR²⁷ was the most effective against GBM cells, killing nearly 90% of the U87_GFP/Luci cells.

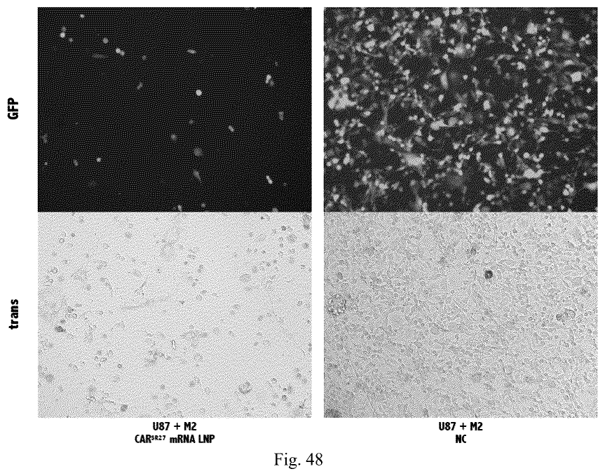

The results were confirmed by direct imaging of GFP in the transfected U87 cells cocultured with M2 macrophages. See FIG. 48.

### Example 18: Identification of GBM Lead TCE_CAR mRNA LNP with High Potency at Very Low E/T Ratio

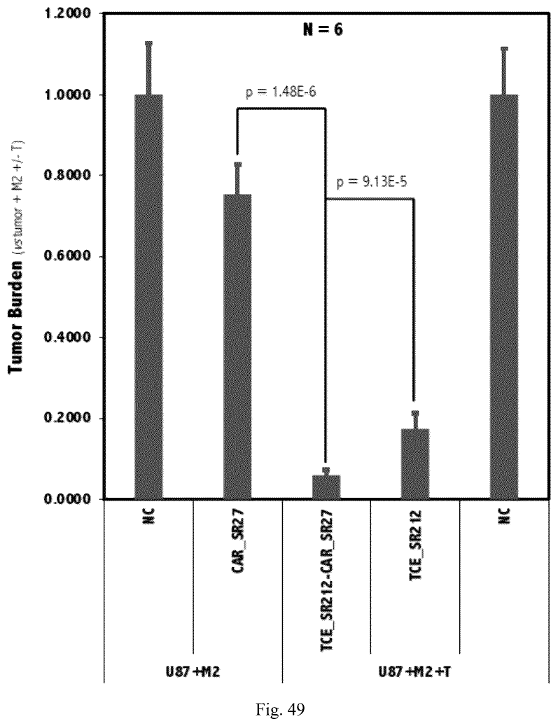

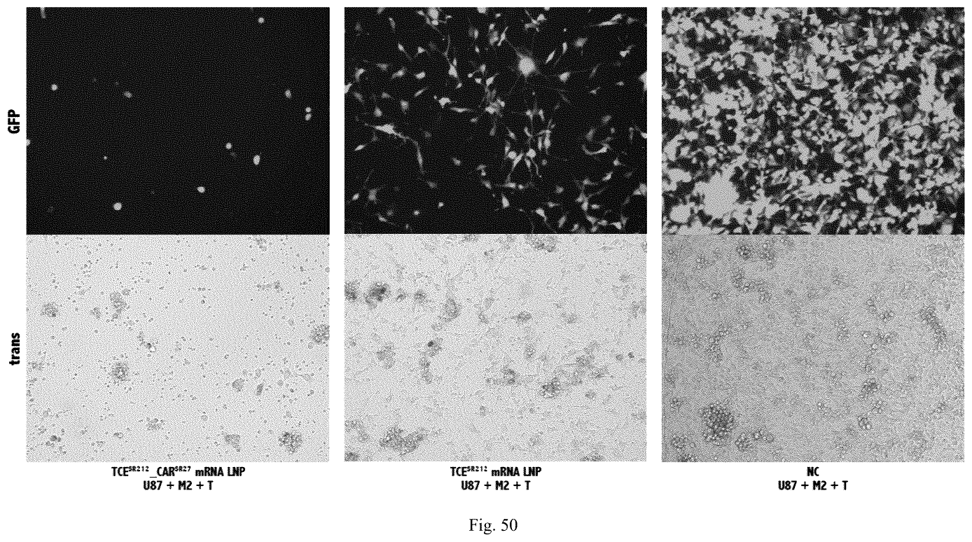

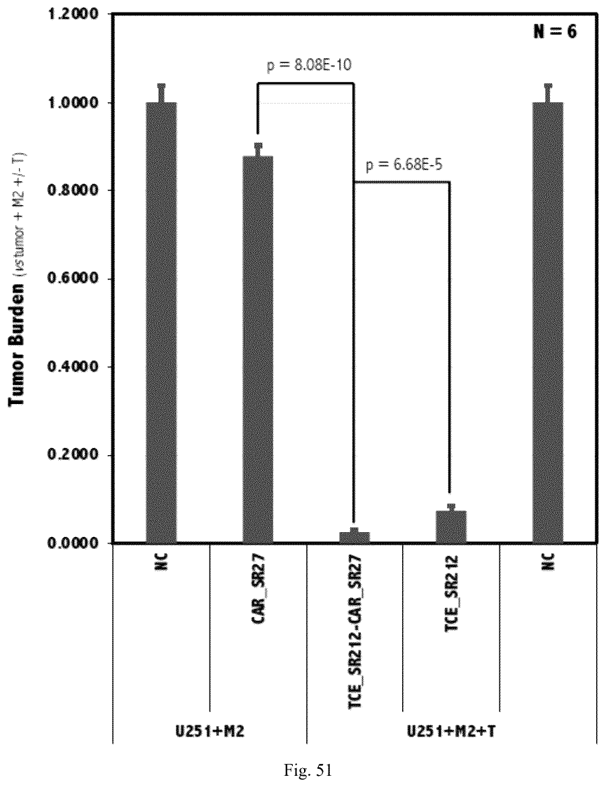

After identification of lead CAR and TCE for treating GBM, both CAR and TCE mRNAs were simultaneously encapsulated into M2 macrophage targeting LNP. The resulting TCE_CAR mRNA LNP (i.e., TCE^{SR212}_CAR^{SR27} mRNA LNP) was tested for its ability to kill GBM cells U87_GFP/Luci cocultured with M2 macrophages and T cells. The results are shown in FIGS. 49 and 50.

Treatment of U87_GFP/Luci/M2/T cell cocultures with TCE^{SR212}_CAR^{SR27} mRNA LNP resulted in significantly more killing of the GBM cells, as compared to TCE^{SR212} mRNA LNP, even though the TCE^{SR212}_CAR^{SR27} mRNA LNP carried half the amount of RNA encoding TCE^{SR212}. See FIG. 49. Treatment with TCESR²¹²_CAR^{SR27} mRNA LNP was also more effective than CAR^{SR27} mRNA LNP treatment of GBM/M2 coculture. See FIG. 49. The effectiveness of TCE^{SR212}_CAR^{SR27} mRNA LNP was even more remarkable given the E/T for M2 of 1/64 and the E/T for T cells of 1/4.

The effectiveness of the TCE_CAR mRNA LNP was confirmed by directly observing GFP expression in the U87_GFP/Luci cells cocultured with M2 and T cells and treated with TCE^{SR212}_CAR^{SR27} mRNA LNP. See FIG. 50.

A similar study showed that the potency of the TCE^{SR212}_CAR^{SR27} mRNA LNP for killing another GBM cell line, i.e., U251_GFP/Luci, cocultured with M2 and T cells (at the same E/T) was significantly better than the TCE mRNA LNP and the CAR mRNA LNP alone. See FIG. 51.

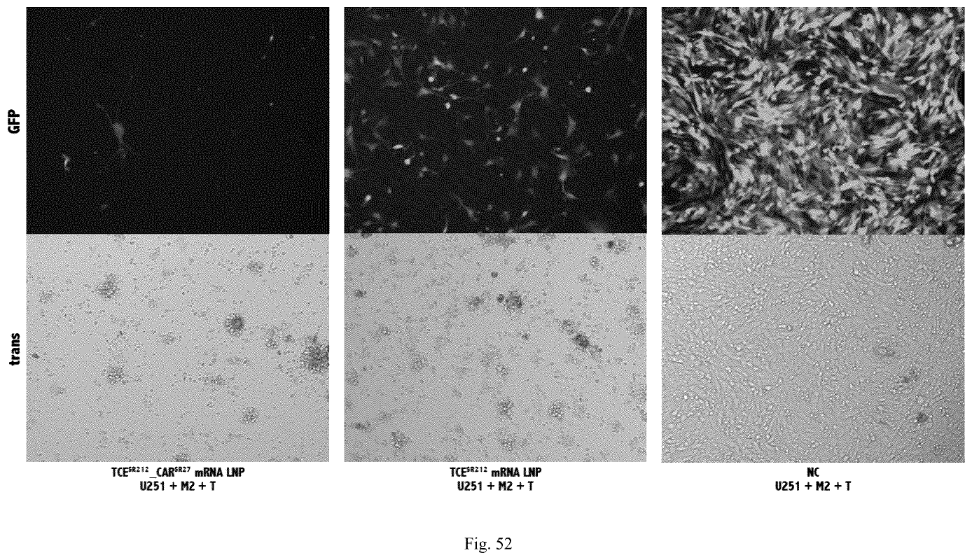

The effectiveness of the TCE_CAR mRNA LNP was confirmed by directly observing GFP expression in the U251_GFP/Luci cells cocultured with M2 and T cells and treated with TCE^{SR212}_CAR^{SR27} mRNA LNP. See FIG. 52.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A nanoparticle for selective transfection of M2 macrophages, the nanoparticle comprising a lipid phase and a core, wherein the core comprises one or more nucleic acids and the nanoparticle selectively delivers the one or more nucleic acids to M2 macrophages in vitro or in vivo.

2. The nanoparticle of claim 1, wherein the lipid phase includes:
an ionizable lipid;
cholesterol or its derivative;
a phospholipid; and
a polyethylene glycol-conjugated lipid (PEGylated lipid), and
the core further comprises a buffer solution.

3. The nanoparticle of claim 2, wherein a mole percentage of the ionizable lipid, the cholesterol or derivative, the phospholipid, and the PEGylated lipid in the nanoparticle is 37% to 47%, 42% to 50%, 7% to 12%, and 1.5% to 5.5%, respectively, and a molar ratio of the ionizable lipid to the one or more nucleic acids is 2:1 to 6:1.

4. The nanoparticle of claim 2, wherein the ionizable lipid is selected from D-Lin-MC3-DMA, SM102, LP01, ALC-0315, and analogs thereof.

5. The nanoparticle of claim 2, wherein the phospholipid is selected from DLPC, DPPC, DSPA, DPPA, DSPC, DMPE, DHPC, DMPC, SPPC, and DAPC.

6. The nanoparticle of claim 5, wherein the PEGylated lipid is DSPE-PEG 2000, DMG-PEG-2000, DSPE-PEG 2000-Mannose, or DMG-PEG-2000-Mannose.

7. The nanoparticle of claim 6, wherein the lipid phase includes at least two PEGylated lipids.

8. The nanoparticle of claim 1, wherein the one or more nucleic acids encode a chimeric antigen receptor (CAR), a T-cell engager (TCE), and, optionally, a therapeutic gene, and the CAR, TCE, and therapeutic gene are expressed in the M2 macrophages.

9. The nanoparticle of claim 8, wherein the CAR has the amino acid sequence of any one of SEQ ID NOs 6 to 16.

10. The nanoparticle of claim 8, wherein the TCE has the amino acid sequence of any one of SEQ ID NOs 1 to 5 and 17 to 21.

11. A method for selective transfection of M2 macrophages, the method comprising contacting M2 macrophages with a composition that includes a plurality of the nanoparticle of claim 1.

12. The method of claim 11, wherein the one or more nucleic acids encode a chimeric antigen receptor (CAR), a T-cell engager (TCE), and/or a therapeutic protein and the CAR, TCE, and/or therapeutic protein are expressed in the M2 macrophages.

13. The method of claim 11, wherein the one or more nucleic acids are mRNA molecules.

14. The method of claim 11, wherein the contacting step is carried out by delivering the composition into a tissue that contains M2 macrophages.

15. The method of claim 14, wherein the tissue is a solid malignant tumor.

16. A therapeutic composition comprising a plurality of the nanoparticle of claim 1 and a pharmaceutically acceptable excipient.

17. A method for converting M2 macrophages into M1 macrophages, the method comprising contacting the M2 macrophages with a plurality of the nanoparticle of claim 1.

18. A method for treating a cancerous tumor in a subject, the method comprising administering to the subject a composition comprising the nanoparticle of claim 8, wherein the CAR specifically binds to a tumor associated antigen (TAA), the TCE specifically binds to another TAA and binds to a T cell, and the therapeutic gene, if present, is effective against the cancerous tumor.

19. The method of claim 18, wherein the therapeutic gene increases the anti-cancer efficacy of the composition, as compared to the combined anti-cancer efficacy of the CAR and TCE.

20. A chimeric antigen receptor (CAR) for treating a tumor, the CAR having the amino acid sequence selected from SEQ ID NOs 6 to 16.

21. An isolated nucleic acid encoding the CAR of claim 20.

22. A T Cell Engager (TCE) for treating a tumor, the TCE having the amino acid sequence selected from SEQ ID NOs 1 to 5 and 17 to 21.

23. An isolated nucleic acid encoding the TCE of claim 22.

24. An isolated nucleic acid for in vitro transcription, the isolated nucleic acid having the sequence of SEQ ID NO: 22.
